(19) United States
(12) Patent Application Publication
(10) Pub. No.: US 2004/0132779 A1
(43) Pub. Date: Jul. 8, 2004

(54) Microsomal Triglyceride transfer protein inhibitor

(75) Inventors: Peter Bertinato, Old Lyme, CT (US); Todd M. Maddux, Foristell, MO (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/742,199

(22) Filed: Dec. 19, 2003

Related U.S. Application Data
(60) Provisional application No. 60/435,378, filed on Dec. 20, 2002.

(51) Int. Cl. A61K 31/444; C 07D 4 1/02

(52) U.S. Cl. 514/332; 514/356; 546/255; 546/315

(57) ABSTRACT

The present invention provides inhibitors of microsomal triglyceride transfer protein (MTP) and/or apolipoprotein B (Apo B) secretion having Formula (I) which are useful for the treatment of obesity and related diseases, as well as prevention and treatment of atherosclerosis and its clinical sequelae, for lowering serum lipids, and in the prevention and treatment of related diseases. The invention further relates to pharmaceutical compositions comprising the compounds of the present invention and to methods of treating obesity, atherosclerosis, and related diseases and/or conditions with the compounds of the present invention, either alone or in combination with other pharmaceutical agents, including lipid-lowering agents.

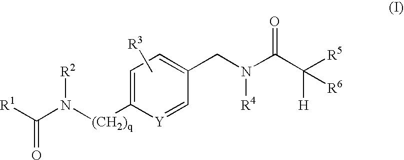

### CROSS REFERENCE TO RELATED APPLICATION

This application claims priority of U.S. Provisional Application No. 60,435,378 filed Dec. 20, 2002.

### FIELD OF THE INVENTION

This application claims priority from U.S. Provisional Patent Application Serial No. 60/435,378, filed Dec. 20, 2002, incorporated herein by reference. This invention relates to inhibitors of microsomal triglyceride transfer protein (MTP) and/or apolipoprotein B (Apo B) secretion which are useful for the treatment of obesity and related diseases, as well as prevention and treatment of atherosclerosis and its clinical sequelae, for lowering serum lipids, and in the prevention and treatment of related diseases. The invention further relates to pharmaceutical compositions comprising these compounds and to methods of treating obesity, atherosclerosis, and related diseases and/or conditions with said compounds, either alone or in combination with other medicaments, including lipid-lowering agents.

### BACKGROUND OF THE INVENTION

Microsomal triglyceride transfer protein catalyzes the transport of triglyceride, cholesteryl ester, and phospholipids and has been implicated as a putative mediator in the assembly of Apo B-containing lipoproteins, biomolecules which contribute to the formation of atherosclerotic lesions. Specifically, the subcellular (lumen of the microsomal fraction) and tissue distribution (liver and intestine) of MTP have led to speculation that it plays a role in the assembly of plasma lipoproteins, as these are the sites of plasma lipoprotein assembly. The ability of MTP to catalyze the transport of triglyceride between membranes is consistent with this speculation, and suggests that MTP may catalyze the transport of triglyceride from its site of synthesis in the endoplasmic reticulum membrane to nascent lipoprotein particles within the lumen of the endoplasmic reticulum.

Accordingly, compounds which inhibit MTP and/or otherwise inhibit Apo B secretion are useful in the treatment of atherosclerosis and other conditions related thereto. Such compounds are also useful in the treatment of other diseases or conditions in which, by inhibiting MTP and/or Apo B secretion, serum cholesterol and triglyceride levels may be reduced. Such conditions may include, for example, hypercholesterolemia, hypertriglyceridemia, pancreatitis, and obesity; and hypercholesterolemia, hypertriglyceridemia, and hyperlipidemia associated with pancreatitis, obesity, and diabetes. For a detailed discussion, see for example, Wetterau et al., Science, 258, 999-1001, (1992), Wetterau et al., Biochem Biophys Acta, 875, 610-617 (1986), European patent application publication Nos. 0 584 446 A2, and 0 643 057 A1, the latter of which refers to certain compounds which have utility as inhibitors of MTP. Other examples of MTP inhibitors may be found in e.g., U.S. Pat. Nos. 5,712,279; 5,741,804; 5,968,950; 6,066,653; and 6,121,283; PCT International Patent Application publications WO 96/40640, WO 97/43257, WO 98/27979, WO 99/33800 and WO 00/05201; and EP 584,446 B and EP 643,057 A.

### SUMMARY OF THE INVENTION

The present invention provides compounds of the Formula (I) having the structure

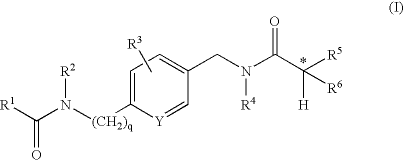

wherein:

R¹ is a group of Formula (IA) having the structure

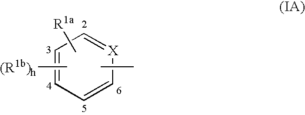

where h is 0 to 3 (preferably, h is 0),

X is N or —C(R^{1c})— (preferably, X is CH),

R^{1a} is phenyl, pyridyl, phenyl-Z—, or pyridyl-Z—, where Z is —S(O)ⱼ—, —O—, —(CR^{1a′}R^{1b′})_{k,} or —(O)ₘ(CR^{1a′}R^{1b′})ₖ(O)ₘ(CR^{1a′}R^{1b′})ₖ—, and the phenyl or pyridyl moieties are optionally substituted with 1 to 3 substituents (preferably, R^{1a} is optionally substituted phenyl, more preferably it is a fluoromethylphenyl, most preferably trifluoromethylphenyl, and wherein the substituent (e.g., F₃C—) is preferably in the para position (e.g., p-trifluoromethylphenyl; where R^{1a} is phenyl-Z—or pyridyl-Z— and Z is —(CR^{1a′}R^{1b′})ₖ— or

—(O)ₘ(CR^{1a′}R^{1b′})ₖ(O)ₘ(CR^{1a′}R^{1b′})ₖ—, Z preferably contains ten or fewer carbon atoms, more preferably eight or fewer carbon atoms, most preferably six or fewer carbon atoms),

R^{1b} and R^{1c} are each independently hydrogen, halo, cyano, nitro, azido, amino, hydroxy, (C₁-C₆)alkyl, (C₂-C₆)alkoxy, methoxy, (C₁-C₆)alkoxy(C₁-C₆)alkyl, -mono-, di- or tri-halo(C₂-C₆)alkyl, perfluoro(C₂-C₄)alkyl, trifluoromethyl, trifluoromethyl(C₁-C₅)alkyl, mono-, di- or tri-halo(C₂-C₆)alkoxy, trifluoromethyl(C₁-C₅)alkoxy, (C₁-C₆)alkylthio, hydroxy(C₁-C₆)alkyl, (C₃-C₈)cycloalkyl(CR^{1a′}R^{1b′})ₖ—, (C₂-C₆)alkenyl, (C₂-C₆)alkynyl, (C₁-C₆)alkylamino-, (C₁-C₆)dialkylamino, amino(C₁-C₆)alkyl-, —(CR^{1a′}R^{1b′})ₖNR^{1a′}R^{1b″}, —C(O)NR^{1b′}R^{1b″}, —NR^{1b″}C(O)R^{1b″′}, —NR^{1b″}OR^{1b″′}, —CH═NOR^{1b″}, —NR^{1b′}C(O)OR^{1b″}, —NR^{1b″}S(O)ⱼR^{1b″}, —C(O)R^{1b″}, —C(S)R^{1b″}, —C(O)OR^{1b″}, —OC(O)R^{1b″}, —SO₂NR^{1b′}R^{1b″}, —S(O)ⱼR^{1b″}, or

—(CR^{1a′}R^{1b′})ₖS(O)ⱼR^{1b″}, where R^{1a′} and R^{1b′} are each independently hydrogen or (C₁-C₆)alkyl, R^{1b″} is H, (C₁-C₆)alkyl, (C₃-C₈)cycloalkyl, —C(O)R^{1b″}, —C(S)R^{1b″′}, —(CR^{1a′}R^{1b′})ₙO(C₁-C₆ alkyl), —(CR^{1a′}R^{1b′})ₙS(C₁-C₆ alkyl), —(CR^{1a′}R^{1b′})ₚC(O)R^{1b″}, —(CR^{1a′}R^{1b′})ₙR^{1b″} or —SO₂R^{1b″}; and

each R^{1b″′} is independently H, (C₁-C₆)alkyl, (C₃-C₈)cycloalkyl, trifluoromethyl, trifluoromethyl(C₁-C₅)alkyl, wherein the alkyl, moieties of the foregoing R^{1b″′} groups are optionally substituted with 1 to 3 substituents each independently selected from the group consisting of C₁-C₆ alkyl, C₁-C₆ alkoxy, amino, hydroxy, halo, cyano, nitro, trifluoromethyl and trifluoromethoxy, j is 0, 1 or 2, each k is independently an integer from 0 to 6, each m is independently 0 or 1, n is an integer from 1 to 6, and p is an integer from 2 to 5 (preferably R^{1b} contains ten or fewer carbon atoms, more preferably eight or fewer carbon atoms, most preferably six or fewer carbon atoms; R^{1c}, independently from R^{1b}, likewise preferably contains ten or fewer carbon atoms, more preferably eight or fewer carbon atoms, most preferably six or fewer carbon atoms, e.g. no carbon atoms);

R is H, (C₁-C₆)alkyl, (C₃-C₈)cycloalkyl, —C(O)R^{1b″}, —C(S)R^{1b″}, —(CR^{1a′}R^{1b′})ₙO(C₁-C₆ alkyl), —(CR^{1a′}R^{1b′})ₙS(C₁-C₆ alkyl), —(CR^{1a′}R^{1b′})ₚC(O)R^{1b″}, —(CR^{1a′}R^{1b′})ₚR^{1b″} or —SO₂R^{1b″}, or R² taken together with R³ forms a 5- to 6-membered partially saturated heterocyclic ring containing one nitrogen atom within the ring (preferably, R² is H or (C₁-C₆)alkyl; more preferably, H or methyl; most preferably, H);

q is 0 or 1 (preferably, q is 0);

R³ is H, halo, (C₁-C₆)alkyl, or mono-, di- or tri-halo(C₁-C₆)alkyl, or R³ taken together with R² forms a 5- to 6-membered partially saturated heterocyclic ring containing one nitrogen atom within the ring (preferably R³ is (C₁-C₆)alkyl, more preferably methyl);

Y is N or C(R³) (preferably, Y is C(CH₃) when R³ is H and CH when R³ is other than H);

R⁴ is H, (C₁-C₆)alkyl, (C₃-C₈)cycloalkyl, —C(O)R^{1b″}, —C(S)R^{1b}″, —(CR^{1a′}R^{1b′})ₙO(C₁-C₆ alkyl), —(CR^{1a′}R^{1b′})ₙS(C₁-C₆ alkyl), —(CR^{1a′}R^{1b′})ₚC(O)R^{1b″}, —(CR^{1a′}R^{1b′})ₚR^{1b″} or —SO₂R^{1b″′}, where n, p, R^{1a′}, R^{1b′} and R^{1b″′} are as defined above (preferably, R⁴ is H or (C₁-C₆)alkyl; more preferably, H or methyl; most preferably, H);

R⁵ is (C₁-C₆)alkyl, an optionally substituted phenyl, or an optionally substituted heteroaryl (preferably, R⁵ is phenyl);

R⁶ is —NH—C(O)—R^{6a} or —NH—C(O)—OR^{6a}, where

R^{6a} is hydrogen, —(CR^{1a′}R^{1b′})ₙO(C₁-C₆ alkyl), —(CR^{1a′}R^{1b′})ₙS(C₁-C₆ alkyl), —(CR^{1a′}R^{1b′})ₚC(O)R^{1b″′}, —(C₁-C₆)alkylSO₂(C₁-C₆)alkyl, —(C₁-C₆)alkylCO₂(C₁-C₆)alkyl, —CH₂O(C₂-C₆)alkylO(C₁-C₆)alkyl, —(C₁-C₆)alkylN(R^{1a′})CO(C₁-C₆)alkyl, —(C₁-C₆)alkylN(R^{1a′})CON(R^{1a′})(R^{1b′}), —(CR^{1a′}R^{1b′})ₚR^{1b′}, or —(CH₂)ₛ—R^{6a′}, where s is an integer from 0 to 6 and R^{6a′} is (C₁-C₆)alkylamino, di(C₁-C₆)alkylamino, or a chemical moiety selected from the group consisting of (C₁-C₆)alkyl, (C₂-C₆)alkenyl, (C₂-C₆)alkynyl, 3- to 6-membered partially or fully saturated carbocyclic ring, 3- to 6-membered partially or fully saturated heterocyclic ring, heteroaryl, and phenyl, where said chemical moiety is optionally substituted with 1 to 3 substituents (preferably R⁶ is —NH—C(O)—R^{6a} and R^{6a} is preferably),

a pharmaceutically acceptable salt thereof or a solvate or hydrate of the compound or the salt.

In a preferred embodiment of the present invention, R¹ is attached at the 2 position of the group of Formula (IA) to provide a compound of Formula (II) having the structure

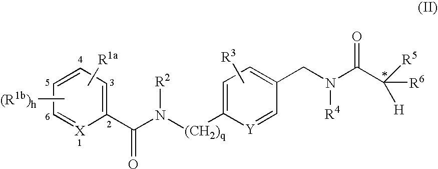

wherein R^{1a}, R^{1b}, h, X, R², q, Y, R³, R⁴, R⁵, and R⁶ are as defined above; a pharmaceutically acceptable salt thereof or a solvate or hydrate of the compound or the salt. Preferably, R^{1a} is attached at the 3 position.

Preferred compounds of the present invention include: (S) 4′-trifluoromethyl-biphenyl-2-carboxylic acid {4-[(2-acetylamino-2-phenyl-acetylamino)-methyl]-2-methyl-phenyl}-amide; (S) 4′-trifluoromethyl-biphenyl-2-carboxylic acid {2-methyl-4-[(2-phenyl-2-propionylamino-acetylamino)-methyl]-phenyl}-amide; (S) 4′-trifluoromethyl-biphenyl-2-carboxylic acid {4-[(2-butyrylamino-2-phenyl-acetylamino)-methyl]-2-methyl-phenyl}-amide; (S) 4′-trifluoromethyl-biphenyl-2-carboxylic acid (2-methyl-4-{[2-phenyl-2-(2,2,2-trifluoro-acetylamino)-acetylamino]-methyl}-phenyl)-amide; (S) 4′-trifluoromethyl-biphenyl-2-carboxylic acid (2-methyl-4-{[2-phenyl-2-(2-m-tolyl-acetylamino)-acetylamino]-methyl}-phenyl)-amide; (S) 4′-trifluoromethyl-biphenyl-2-carboxylic acid {4-[(2-phenyl-2-propionylamino-acetylamino)-methyl]-phenyl}-amide; (S) 4′-trifluoromethyl-biphenyl-2-carboxylic acid {2-methyl-4-[(2-pentanoylamino-2-phenyl-acetylamino)-methyl]-phenyl}-amide; (S) 4′-trifluoromethyl-biphenyl-2-carboxylic acid {4-[(2-butyrylamino-2-phenyl-acetylamino)-methyl]-2-chloro-phenyl}-amide; (S) 4′-trifluoromethyl-biphenyl-2-carboxylic acid [4-({2-[2-(3-chloro-phenyl)-acetylamino]-2-phenyl-acetylamino}-methyl)-2-methyl-phenyl]-amide; (S) 4′-trifluoromethyl-biphenyl-2-carboxylic acid [4-({2-[3-(4-methoxy-phenyl)-propionylamino]-2-phenyl-acetylamino}-methyl)-2-methyl-phenyl]-amide; (S) 4′-trifluoromethyl-biphenyl-2-carboxylic acid (2-chloro-4-{[2-phenyl-2-(2,2,2-trifluoro-acetylamino)-acetylamino]-methyl}-phenyl)-amide; (S) 4′-trifluoromethyl-biphenyl-2-carboxylic acid {4-[(2-pentanoylamino-2-phenyl-acetylamino)-methyl]-phenyl}-amide; (S) 4′-trifluoromethyl-biphenyl-2-carboxylic acid [4-({2-[2-(3-fluoro-phenyl)-acetylamino]-2-phenyl-acetylamino}-methyl)-2-methyl-phenyl]-amide; (S) 4′-trifluoromethyl-biphenyl-2-carboxylic acid [4-({2-[2-(4-ethoxy-phenyl)-acetylamino]-2-phenyl-acetylamino}-methyl)-2-methyl-phenyl]-amide; (S) 4′-trifluoromethyl-biphenyl-2-carboxylic acid (2-methyl-4-{[2-(2-naphthalen-1-yl-acetylamino)-2-phenyl-acetylamino]-methyl}-phenyl)-amide; (S) 4′-trifluoromethyl-biphenyl-2-carboxylic acid (4-{[2-(2-methoxy-acetylamino)-2-phenyl-acetylamino]-methyl}-phenyl)-amide; (S) 4′-trifluoromethyl-biphenyl-2-carboxylic acid (2-methyl-4-{[2-phenyl-2-(4-phenyl-butyrylamino)-acetylamino]-methyl}-phenyl)-amide; (S) 4′-trifluoromethyl-biphenyl-2-carboxylic acid (4-[2-(2-methoxy-acetylamino)-2-phenyl-acetylamino]-methyl)-2-methyl-phenyl)-amide; (S) 4′-trifluoromethyl-biphenyl-2-carboxylic acid (2-chloro-4-{[2-(2-chloro-acetylamino)-2-phenyl-acetylamino]-methyl}-phenyl)-amide; and (S) 4′-trifluoromethyl-biphenyl-2-carboxylic acid (2-methyl-4-{[2-(2,2,3,3,3-pentafluoro-propionylamino)-2-phenyl-acetylamino]-methyl}-phenyl)-amide; a pharmaceutically acceptable salt thereof, a prodrug of said compound or said salt, or a solvate or hydrate of said compound, said salt or said prodrug.

Some of the compounds described herein contain at least one chiral center; consequently, those skilled in the art will appreciate that all stereoisomers (e.g., enantiomers and diasteroisomers) of the compounds illustrated and discussed herein are within the scope of the present invention. In addition, tautomeric forms of the compounds are also within the scope of the present invention. When R⁵ is phenyl, the carbon atom to which R⁵ is attached (e.g., the carbon indicated with an asterick in the compound of Formula (I) or (II) above) is preferably in the (S) configuration.

In another embodiment of the present invention, a pharmaceutical composition is provided that comprises (1) a compound of the present invention; and (2) a pharmaceutically acceptable excipient, diluent, or carrier. Preferably, the composition comprises a therapeutically effective amount of a compound of the present invention. The composition may also contain at least one additional pharmaceutical agent (described herein). Preferred agents include lipid-lowering agents, cholesterol absorption inhibitors, CETP inhibitors, HMG-CoA reductase inhibitors, HMG-CoA synthase inhibitors, inhibitors of HMG-CoA reductase gene expression, niacin, antioxidants, ACAT inhibitors, PPAR inhibitors, squalene synthetase inhibitors, and anti-obesity agents.

In yet another embodiment of the present invention, a method is provided for treating a disease, condition or disorder modulated by the inhibition of a microsomal triglyceride transfer protein and/or apolipoprotein B secretion in animals which comprises administering to an animal in need of such treatment a therapeutically effective amount of a compound of the present invention (or a pharmaceutical composition thereof).

Diseases, conditions, and/or disorders modulated by microsomal triglyceride transfer protein and/or apolipoprotein B secretion include atherosclerosis, pancreatitis, obesity and weight management (including conditions in which weight loss, food intake reduction, etc. are desired), hypercholesterolemia, hypertriglyceridemia, hyperlipidemia, and diabetes. In one embodiment, a method is provided for treating atherosclerosis; pancreatitis secondary to hypertriglyceridemia or hyperglycemia (1) by causing a reduced absorption of dietary fat through MTP inhibition, (2) by lowering triglycerides through MTP inhibition or (3) by decreasing the absorption of free fatty acids through MTP inhibition, which comprises administering to an animal in need of such treatment a therapeutically effective amount of a compound of the present invention.

In another embodiment, a method is provided for treating diabetes in an animal, which comprises administering to an animal in need of such treatment a therapeutically effective amount of a compound of the present invention.

In yet another embodiment, a method is provided for treating obesity in an animal, which comprises administering to an animal in need of such treatment a therapeutically effective amount of a compound of the present invention.

In another aspect of the present invention, a combination therapy is provided where a compound of the present invention is administered in combination with other pharmaceutical agents. Preferred pharmaceutical agents include lipid-lowering agents, cholesterol absorption inhibitors, PPAR inhibitors, CETP inhibitors, HMG-CoA reductase inhibitors, HMG-CoA synthase inhibitors, inhibitors of HMG-CoA reductase gene expression, niacin, antioxidants, ACAT inhibitors, squalene synthetase inhibitors, and anti-obesity agents such as cannabinoid antagonists or reverse agonists, peptide YY and agonists thereof (e.g. peptide YY₃₋₃₆), MCR-4 agonists, CCK-A agonists, monoamine reuptake inhibitors, sympathomimetic agents, β₃ adrenergic receptor agonists, dopamine agonists, melanocyte-stimulating hormone receptor analogs, 5-HT2c receptor agonists, melanin concentrating hormone antagonists, leptin, leptin analogs, leptin receptor agonists, galanin antagonists, lipase inhibitors, bombesin agonists, neuropeptide-Y antagonists, thyromimetic agents, dehydroepiandrosterone or analogs thereof, glucocorticoid receptor antagonists, orexin receptor antagonists, glucagon-like peptide-1 receptor agonists, ciliary neurotrophic factors, human agouti-related protein antagonists, ghrelin receptor antagonists, histamine 3 receptor antagonists or inverse agonists, and neuromedin U receptor agonists, and the like.

The combination therapy may be administered to animal in need of such treatment as (a) a single pharmaceutical composition which comprises a compound of the present invention, at least one additional pharmaceutical agent described herein and a pharmaceutically acceptable excipient, diluent, or carrier; or (b) two separate pharmaceutical compositions comprising (i) a first composition comprising a compound of the present invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

### Definitions

As used herein, the term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like). Similarly, the alkyl portion (i.e., alkyl moiety) of an alkoxy, acyl (e.g., alkanoyl), alkylamino, dialkylamino, and alkylthio group have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls) independently selected from the group of substituents listed below in the definition for “substituted.” “Halo-substituted alkyl” refers to an alkyl group substituted with one or more halogen atoms (e.g., fluoromethyl, difluoromethyl, trifluoromethyl, perfluoroethyl, and the like). Preferably, alkyl moieties comprising a CH₃ (methyl), CH₂ (methylene), or CH (methine) group which is not substituted with halogen, SO or SO₂, or attached to a N, O or S atom may optionally bear on the methyl, the methylene or the methine group a substituent selected halo, —OR^{1a′}, —SR^{1a′} or —NR^{1a′}R^{1b′} where R^{1a′} and R^{1b′} are as defined above.

The term “alkenyl” refers to both straight and branched chain hydrocarbon groups containing at least two carbons and at least one unsaturation within the chain. Some examples of alkenyl groups are ethenyl, propenyl, isobutenyl, 1,3-pentadienyl, 2,4-pentadienyl, and the like. Preferably, alkenyl moieties comprising a CH₃ (methyl), CH₂ (methylene), or CH (methine) group which is not substituted with halogen, SO or SO₂, or attached to a N, O or S atom may optionally bear on the methyl, the methylene or the methine group a substituent selected halo, —OR^{1a′}, —SR^{1a′} or —NR^{1a′}R^{1b′} where R^{1a′} and R^{1b′} are as defined above.

The term “alkynyl” means both straight and branched chain hydrocarbon groups containing at least one triple bond between two carbon atoms. Some examples of alknyl groups are ethynyl and propynyl, e.g., propyn-1-yl and propyn-2-yl and propyn-3-yl. Preferably, alkynyl moieties comprising a CH₃ (methyl), CH₂ (methylene), or CH (methine) group which is not substituted with halogen, SO or SO₂, or attached to a N, O or S atom may optionally bear on the methyl, the methylene or the methine group a substituent selected halo, —OR^{1a′}, —SR^{1a′} or —NR^{1a′}R^{1b′} where R^{1a′} and R^{1b′} are as defined above.

The terms “partially or fully saturated carbocyclic ring” (also referred to as “partially or fully saturated cycloalkyl”) refers to nonaromatic rings that are either partially or fully hydrogenated and may exist as a single ring, bicyclic ring or a spiro-fused ring. Unless specified otherwise, the carbocyclic ring is generally a 3- to 8-membered ring. For example, partially or fully saturated carbocyclic rings (or cycloalkyl) include groups such as cyclopropyl, cyclopropenyl, cyclobutyl, cyclobutenyl, cyclopentyl, cyclpentenyl, cyclopentadienyl, cyclohexyl, cyclohexenyl, cyclohexadienyl, norbornyl (bicyclo[2.2.1]heptyl), norbornenyl, bicyclo[2.2.2]octyl, and the like. When designated as being “optionally substituted”, the partially saturated or fully saturated cycloalkyl group may be unsubstituted or substituted with one or more substituents (typically, one to three substituents) independently selected from the group of substituents listed below in the definition for “substituted.” A substituted carbocyclic ring also includes groups wherein the carbocyclic ring is fused to a phenyl ring (e.g., indanyl). The carbocyclic group may be attached to the chemical entity or moiety by any one of the carbon atoms within the carbocyclic ring system. When substituted, the carbocyclic group is preferably substituted with 1 or 2 substituents independently selected from carboxy (—CO₂H), aminocarbonyl (—CONH₂), mono- or di-(C₁-C₆)alkylaminocarbonyl (mono- or di-(C₁-C₆)alkylamino-C(O)—), acyl, (C₁-C₃)alkyl, (C₂-C₃)alkenyl, (C₁-C₆)alkynyl, aryl, heteroaryl, 3- to 6-membered heterocycle, chloro, fluoro, cyano, hydroxy, (C₁-C₃)alkoxy, aryloxy, heteroaryloxy, acyloxy, amino, (C₁-C₆)alkylamino, di-(C₁-C₄)alkylamino, carbamoyl (i.e., (C₁-C₃)alkyl-O—C(O)—NH— or mono- or di-(C₁-C₃)alkylamino-C(O)—O—), (C₁-C₆)alkoxycarbonyl, (C₃-C₆)cycloalkoxycarbonyl, aryloxycarbonyl, heteroaryloxycarbonyl, hydroxy(C₂-C₃)alkylamino, or oxo, wherein each aminocarbonyl, mono- or di-alkylaminocarbonyl, acyl, alkyl, cycloalkyl, alkenyl, alkynyl, aryl, heteroaryl, heterocycle, alkoxy, aryloxy, heteroaryloxy, acyloxy, alkylamino, dialkylamino, carbamoyl, alkoxycarbonyl, cycloalkoxycarbonyl, aryloxycarbonyl, heteroaryloxycarbonyl and hydroxyalkylamino can be optionally substituted with up to three substituents independently selected from chlorine, fluorine, hydroxy, cyano, and amino, and more preferably 1 or 2 from substituents independently selected from (C₁-C₂)alkyl, 3- to 6-membered heterocycle, fluoro, (C₁-C₃)alkoxy, (C₁-C₄)alkylamino or di-(C₁-C₂)alkylamino optionally substituted as described above. Similarly, any cycloalkyl portion of a group (e.g., cycloalkylalkyl, cycloalkylamino, etc.) has the same definition as above.

The term “partially saturated or fully saturated heterocyclic ring” (also referred to as “partially saturated or fully saturated heterocycle”) refers to nonaromatic rings that are either partially or fully hydrogenated and may exist as a single ring, bicyclic ring or a spiro-fused ring. Unless specified otherwise, the heterocyclic ring is generally a 3- to 6-membered ring containing 1 to 3 heteroatoms (preferably 1 or 2 heteroatoms) independently selected from sulfur, oxygen and/or nitrogen. Partially saturated or fully saturated heterocyclic rings include groups such as epoxy, aziridinyl, tetrahydrofuranyl, dihydrofuranyl, dihydropyridinyl, pyrrolidinyl, N-methylpyrrolidinyl, imidazolidinyl, imidazolinyl, piperidinyl, piperazinyl, pyrazolidinyl, 2H-pyranyl, 4H-pyranyl, 2H-chromenyl, oxazinyl, morpholino, thiomorpholino, tetrahydrothienyl, tetrahydrothienyl 1,1-dioxide, and the like. When indicated as being “optionally substituted”, the partially saturated or fully saturated heterocycle group may be unsubstituted or substituted with one or more substituents (typically, one to three substituents) independently selected from the group of substituents listed below in the definition for “substituted.” A substituted heterocyclic ring includes groups wherein the heterocyclic ring is fused to an aryl or heteroaryl ring (e.g., 2,3-dihydrobenzofuranyl, 2,3-dihydroindolyl, 2,3-dihydrobenzothiophenyl, 2,3-dihydrobenzothiazolyl, etc.). When substituted, the heterocycle group is preferably substituted with 1 or 2 substituents independently selected from acyl, (C₁-C₃)alkyl, (C₃-C₆)cycloalkyl, (C₂-C₄)alkenyl, (C₁-C₆)alkynyl, aryl, heteroaryl, 3- to 6-membered heterocycle, chloro, fluoro, cyano, hydroxy, (C₁-C₃)alkoxy, aryloxy, heteroaryloxy, acyloxy, amino, (C₁-C₆)alkyl amino, di-(C₁-C₃)alkyl amino, carbamoyl (i.e., (C₁-C₃)alkyl-O—C(O)—NH— or mono- or di-(C₁-C₃)alkylamino-C(O)—O—), (C₁-C₆)alkoxycarbonyl, (C₃-C₆)cycloalkoxycarbonyl, aryloxycarbonyl, heteroaryloxycarbonyl, hydroxy(C₂-C₃)alkylamino, or oxo, wherein each aminocarbonyl, mono- or di-alkylaminocarbonyl, acyl, alkyl, cycloalkyl, alkenyl, alkynyl, aryl, heteroaryl, heterocycle; alkoxy, aryloxy, heteroaryloxy, acyloxy, alkylamino, dialkylamino, carbamoyl, alkoxycarbonyl, cycloalkoxycarbonyl, aryloxycarbonyl, heteroaryloxycarbonyl and hydroxyalkylamino can be optionally substituted with up to three substituents independently selected from chlorine, fluorine, hydroxy, cyano, and amino, and more preferably with 1 or 2 substituents independently selected from (C₁-C₃)alkyl, (C₃-C₆)cycloalkyl, (C₆)aryl, 6-membered heteroaryl, 3- to 6-membered heterocycle, or fluoro. The heterocyclic group may be attached to the chemical entity or moiety by any one of the ring atoms within the heterocyclic ring system. Similarly, any heterocycle portion of a group (e.g., heterocycle-substituted alkyl, heterocycle-substituted carbonyl, etc.) has the same definition as above.

The term “aryl” or “aromatic carbocyclic ring” refers to aromatic moieties having a single (e.g., phenyl) or a fused ring system (e.g., naphthalene, anthracene, phenanthrene, etc.). A typical aryl group is a 6- to 10-membered aromatic carbocyclic ring(s). A preferred aryl group is phenyl. When indicated as being “optionally substituted”, the aryl groups (including an optionally substituted phenyl) may be unsubstituted or substituted with one or more substituents (preferably no more than three substituents) independently selected from the group of substituents listed below in the definition for “substituted.” Substituted aryl groups include a chain of aromatic moieties (e.g., biphenyl, terphenyl, phenylnaphthyl, etc.). When substituted, the aromatic moieties are preferably substituted with 1 or 2 substituents independently selected from carboxy (—CO₂H), aminocarbonyl (—CONH₂), mono- or di-(C₁-C₆)alkylaminocarbonyl(mono- or di-(C₁-C₆)alkylamino-C(O)—), acyl, (C₁-C₄)alkyl, (C₃-C₆)cycloalkyl, (C₂-C₃)alkenyl, (C₁-C₆)alkynyl, aryl, heteroaryl, 3- to 6-membered heterocycle, bromo, chloro, fluoro, iodo, cyano, hydroxy, (C₁-C₄)alkoxy, aryloxy, heteroaryloxy, acyloxy, amino, (C₁-C₆)alkylamino, di-(C₁-C₃)alkylamino, hydroxy(C₂-C₃)alkylamino, (C₁-C₆)alkoxycarbonyl, (C₃-C₆)cycloalkoxycarbonyl, aryloxycarbonyl, heteroaryloxycarbonyl, or carbamoyl (i.e., (C₁-C₃)alkyl-O—C(O)—NH— or mono- or di-(C₁-C₃)alkylamino-C(O)—O—), wherein each aminocarbonyl, mono- or di-alkylaminocarbonyl, acyl, alkyl, cycloalkyl, alkenyl, alkynyl, aryl, heteroaryl, heterocycle, alkoxy, aryloxy, heteroaryloxy, acyloxy, alkylamino, dialkylamino, carbamoyl, alkoxycarbonyl, cycloalkoxycarbonyl, aryloxycarbonyl, heteroaryloxycarbonyl and hydroxyalkylamino can be optionally substituted with up to three substituents independently selected from chlorine, fluorine, hydroxy, cyano, and amino, and more preferably, 1 or 2 substituents independently selected from (C₁-C₄)alkyl, chloro, fluoro, cyano, hydroxy, or (C₁-C₄)alkoxy optionally substituted as described above. The aryl group may be attached to the chemical entity or moiety by any one of the carbon atoms within the aromatic ring system. Similarly, the aryl portion (i.e., aromatic moiety) of an aroyl or aroyloxy (i.e., (aryl)-C(O)—O—) has the same definition as above.

The term “heteroaryl” or “heteroaromatic ring” refers to aromatic moieties containing at least one heteratom (e.g., oxygen, sulfur, nitrogen or combinations thereof) within a 5- to 10-membered aromatic ring system (e.g., pyrrolyl, pyridyl, pyrazolyl, indolyl, indazolyl, thienyl, furanyl, benzofuranyl, oxazolyl, imidazolyl, tetrazolyl, triazinyl, pyrimidyl, pyrazinyl, thiazolyl, purinyl, benzimidazolyl, quinolinyl, isoquinolinyl, benzothiophenyl, benzoxazolyl, etc.). The heteroaromatic moiety may consist of a single or fused ring system. A typical single heteroaryl ring is a 5- to 6-membered ring containing one to three heteroatoms independently selected from oxygen, sulfur and nitrogen and a typical fused heteroaryl ring system is a 9- to 10-membered ring system containing one to four heteroatoms independently selected from oxygen, sulfur and nitrogen. When indicated as being “optionally substituted”, the heteroaryl groups may be unsubstituted or substituted with one or more substituents (preferably no more than three substituents) independently selected from the group of substituents listed below in the definition for “substituted.” When substituted, the heteroaromatic moieties are preferably substituted with 1 or 2 substituents independently selected from carboxy (—CO₂H), aminocarbonyl (—CONH₂), mono- or di-(C₁-C₆)alkylaminocarbonyl (mono- or di-(C₁-C₆)alkylamino-C(O)—), acyl, (C₁-C₄)alkyl, (C₃-C₆)cycloalkyl, (C₂-C₃)alkenyl, (C₁-C₆)alkynyl, aryl, heteroaryl, 3- to 6-membered heterocycle, bromo, chloro, fluoro, iodo, cyano, hydroxy, (C₁-C₄)alkoxy, aryloxy, heteroaryloxy, acyloxy, amino, (C₁-C₆)alkylamino, di-(C₁-C₃)alkylamino, hydroxy(C₂-C₃)alkylamino, (C₁-C₆)alkoxycarbonyl, (C₃-C₆)cycloalkoxycarbonyl, aryloxycarbonyl, heteroaryloxycarbonyl, or carbamoyl (i.e., (C₁-C₃)alkyl-O—C(O)—NH— or mono- or di-(C₁-C₃)alkylamino-C(O)—O—), wherein each aminocarbonyl, mono- or di-alkylaminocarbonyl, acyl, alkyl, cycloalkyl, alkenyl, alkynyl, aryl, heteroaryl, heterocycle, alkoxy, aryloxy, heteroaryloxy, acyloxy, alkylamino, dialkylamino, carbamoyl, alkoxycarbonyl, cycloalkoxycarbonyl, aryloxycarbonyl, heteroaryloxycarbonyl and hydroxyalkylamino can be optionally substituted with up to three substituents independently selected from chlorine, fluorine, hydroxy, cyano, and amino, and more preferably, 1 or 2 substituents independently selected from (C₁-C₄)alkyl, chloro, fluoro, cyano, hydroxy, (C₁-C₄)alkoxy, (C₁-C₄)alkyl amino or di-(C₁-C₂)alkyl amino optionally substituted as described above. The heteroaryl group may be attached to the chemical entity or moiety by any one of the atoms within the aromatic ring system (e.g., imidazol-1-yl, imidazol-2-yl, imidazol-4-yl, imidazol-5-yl, pyrid-2-yl, pyrid-3-yl, pyrid-4-yl, pyrid-5-yl, or pyrid-6-yl). Similarly, the heteroaryl portion (i.e., heteroaromatic moiety) of a heteroaroyl (i.e., (heteroaryl)-C(O)—O—) has the same definition as above.

The term “acyl” refers to formyl as well as alkyl, alkenyl, alkynyl, partially saturated or fully saturated cycloalkyl, partially saturated or fully saturated heterocycle, aryl, and heteroaryl substituted carbonyl groups. For example, acyl includes groups such as (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions above. When indicated as being “optionally substituted”, the acyl group may be unsubstituted or optionally substituted with one of more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “halo” or “halogen” refers to chlorine, bromine, iodine and fluorine.

The term “substituted” specifically envisions and allows for one or more substitutions that are common in the art. However, it is generally understood by those skilled in the art that the substituents should be selected so as to not adversely affect the pharmacological characteristics or stability of the compound or adversely interfere with the use of the medicament. Suitable substituents for any of the groups defined above include (C₁-C₆)alkyl, (C₃-C₇)cycloalkyl, (C₂-C₆)alkenyl, (C₁-C₆)alkynyl, aryl, heteroaryl, 3- to 6-membered heterocycle, halo (e.g., chloro, bromo, iodo and fluoro), cyano, hydroxy, (C₁-C₆)alkoxy, aryloxy, heteroaryloxy, sulfhydryl (mercapto), (C₁-C₆)alkylthio, arylthio, heteroarylthio, amino, mono- or di-(C₁-C₆)alkylamino, quaternary ammonium salts, amino(C₁-C₆)alkoxy, carbamoyl (i.e., (C₁-C₆)alkyl-O—C(O)—NH— or mono- or di-(C₁-C₃)alkylamino-C(O)—O—), hydroxy(C₂-C₆)alkylamino, amino(C₁-C₆)alkylthio, nitro, oxo, acyl, (C₁-C₆)alkyl-CO₂—, glycolyl, glycyl, hydrazino, guanyl, thio(C₁-C₆)alkyl-C(O)—, thio(C₁-C₆)alkyl-CO₂—, and combinations thereof. In the case of substituted combinations, such as “substituted aryl(C₁-C₆)alkyl”, either the aryl or the alkyl group may be substituted, or both the aryl and the alkyl groups may be substituted with one or more independently selected substituents (typically, one to three substituents except in the case of perhalo substitutions). An aryl or heteroaryl substituted carbocyclic or heterocyclic group may be a fused ring (e.g., indanyl, dihydrobenzofuranyl, dihydroindolyl, etc.).

The term “solvate” refers to a molecular complex of a compound represented by Formula (I) or (II) (including prodrugs and pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male and female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The term “compounds of the present invention” (unless specifically identified otherwise) refer to compounds of Formula (I) and (II), prodrugs thereof, pharmaceutically acceptable salts of the compounds, and/or prodrugs, and hydrates or solvates of the compounds, salts, and/or prodrugs, as well as, all stereoisomers (including diastereoisomers and enantiomers), tautomers and isotopically labeled compounds.

### DETAILED DESCRIPTION

The present invention provides compounds and pharmaceutical formulations thereof that are useful in the treatment of diseases linked to the inhibition of the microsomal triglyceride transfer protein (MTP) and/or apolipoprotein B (Apo B) secretion.

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well-known to those skilled in the art.

In the preparation of compounds of the present invention, protection of remote functionality (e.g., primary or secondary amine or carboxylic acid) of intermediates may be necessary. The need for such protection will vary depending on the nature of the remote functionality and the conditions of the preparation methods. Suitable amino-protecting groups (NH—Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Compounds of the present invention may be prepared using analogous procedures and starting materials described in U.S. patent application Ser. No. 10/177,858 entitled “Triamide-Substituted Heterocyclic Compounds,” filed on Jun. 20, 2002, and incorporated herein by reference. In general, the compounds of the present invention may be prepared by forming amide linkages between compounds having the following general structures A, B and C.

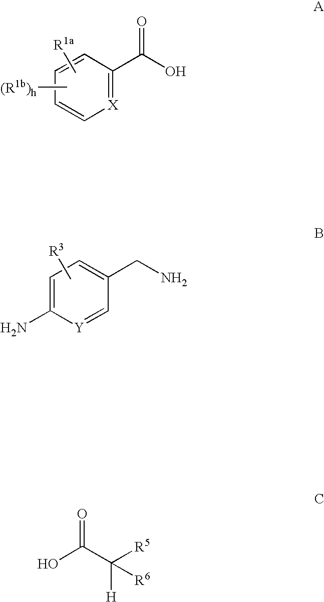

Compounds A, B and C are either commercially available or readily prepared using procedures well-known to those skilled in the art. For example, preferred compounds of Formula A where X is —C(R^{1c})— and R^{1a} is an optionally substituted phenyl are commercially available (e.g., 2-biphenylcarboxylic acid, 4′-methyl-2-biphenylcarboxylic acid and 4′-trifluoromethyl-2-biphenylcarboxylic). In addition, numerous pyridyl-phenyl (X is nitrogen and R^{1a} is phenyl or a substituted phenyl) and bipyridyl (X is nitrogen and R^{1a} is pyridyl) compounds are also readily obtained either commercially or by derivatization of commercial materials. Preferred compounds of Formula B may be readily prepared from their corresponding nitro-substituted compounds (e.g., p-nitronicotinic acid, p-nitrobenzoic acid, and derivatives thereof). Preferred compounds of Formula C where R⁵ is an optionally substituted phenyl and R⁶ is —NHC(O)R^{6a} are readily prepared from commercially available phenyl glycines (both S and R configurations), where the amide moiety —NHC(O)R^{6a} is formed between the amino group of the phenylglycine and the carboxylic acid HO—C(O)R^{6a} or activated carboxylic acid L-C(O)R^{6a}, where L is a leaving group (e.g., chloride) or a carbonic acid monoester derivative (e.g., HO—C(O)OR^{6a}).

Scheme I below illustrates one means for preparing compounds of the present invention, where R³, R^{1a}, R^{1b}, h, Y, X, R⁵ and R^{6a} are as defined above and Pg is a protecting group.

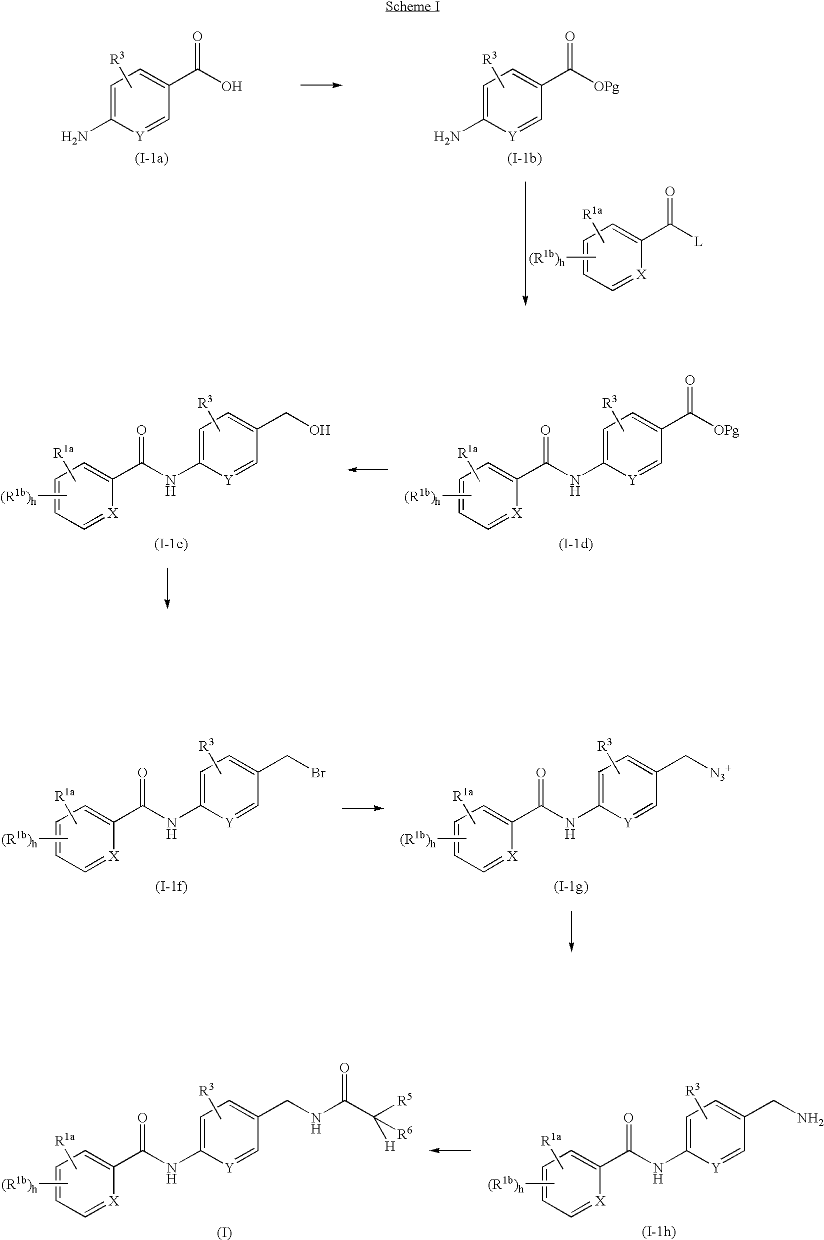

The aminophenylcarboxylic acid (I-1a) is commercially available or readily prepared from commercially available materials using conventional procedures well-known to those skilled in the art (e.g., reduction of the corresponding commercially available nitro compounds (e.g., p-nitronicotinic acid, p-nitrobenzoic acid and derivatives thereof) via catalytic hydrogenation). Before coupling the amino compound (I-1a) with the activated carboxylic acid (I-1c), the carboxylic acid functionality of intermediate (I-1a) is protected using standard carboxylic acid protection procedures, e.g., formation of the corresponding ester. The activated carboxylic acid (I-1c) can be readily prepared using materials and methods that are well-known in the art. For example, the acid chloride compounds (I-1c) where X is —C(R^{1c})— and R^{1a} is an optionally substituted phenyl may be prepared from the corresponding commercially available carboxylic acids (e.g., 2-biphenylcarboxylic acid, 4′-methyl-2-biphenylcarboxylic acid and 4′trifluoromethyl-2-biphenylcarboxylic) using procedures well-known to those skilled in the art (e.g., treatment with oxalyl chloride or sulfonyl chloride). The amide (I-1d) is then formed by coupling the acid chloride (I-1c) with the amino compound (I-1b). The ester can be reduced to the alcohol using conventional reducing agents (e.g. LiBH₄).(I-1e). The hydroxy group of intermediate (I-1 e) is converted to an amino group by first substituting the hydroxy with a halogen (e.g., bromine) using conventional halogenation procedures (e.g., PBr₃ in 0° C. under anhydrous conditions) to form the halogenated compound (I-1f). The bromine is then replace with an azide to form the azo compound (I-1g) followed by reduction of the azide to the amine to produce the amino compounds (I-1 h). The final amide linkage may then be accomplished by acylating the amino functionality of compound (I-1 h) with the desired activated carboxylic acid or carbonic acid derivative to produce a compound of Formula (I).

The conversion of compound (I-1 h) into final product is preferably conducted in two steps: treatment of compound (I-1 h) with Boc-protected HO₂C CH(R⁵)NH₂ to produce, after removal of the protecting group, the —C(O)CH(R⁵)NH₂ adduct of compound (I-1 h) which on treatment with the appropriate, activated HO₂C—R^{6a} or HO₂C—OR^{6a} gives the compound of Formula (I).

For a more detailed description of the reaction conditions, see the Examples below.

Intermediate (I-1 h) may also be prepared from the acid halide or other activated derivative of compound (I-1a) in three steps as illustrated in Scheme II. Also illustrated in Scheme II is the 2-step conversion of compound I-1h into the compound of Formula (I).

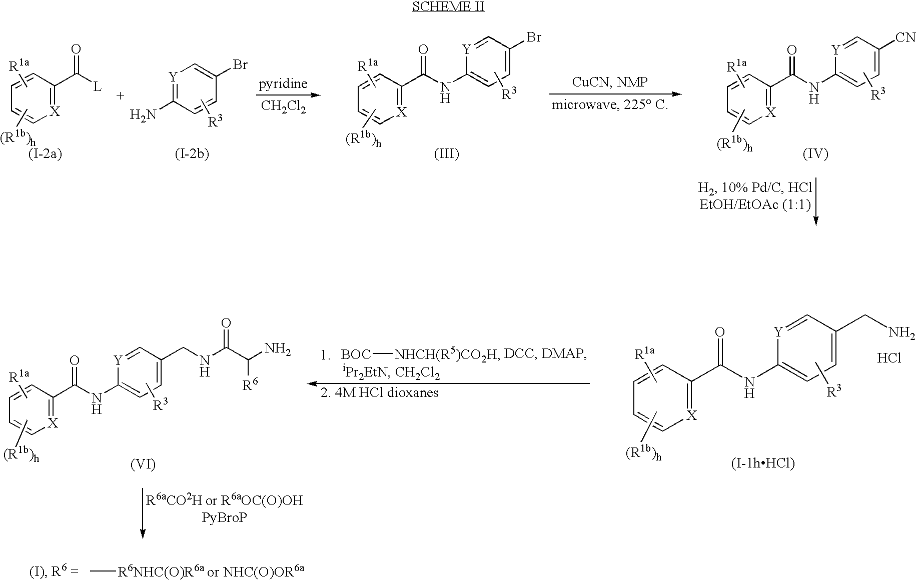

The leaving group “L” in compound (I-2a) is preferably a chlorine atom, but may be any leaving group useful in amidation reactions. The bromo(pyridyl or phenyl)amine, compound (I-2b), is commercially available or may be prepared by methods known in the art from readily available starting materials. Coupling of compounds (I-2a) and (I-2b) is conducted in the presence of base (e.g., pyridine) in an organic solvent (e.g., CH₂Cl₂) to give the corresponding amide, compound (III). Bromo compound (III) is converted into the corresponding cyano compound, compound (IV), upon treatment with CuCN in a microwave reactor at elevated temperature in the presence of an appropriate organic solvent (e.g., N-methylpyrrolidine). Compound (IV) is catalytically reduced in the presence of acid (e.g., HCl) to give the corresponding salt of compound (I-1-h). Compound (I-1h.HCl) is treated with an appropriate Boc-protected amino acid (e.g. Boc-phg-OH) in the presence of a base (e.g., diisopropylethylamine), a coupling agent (e.g., DCC) and a catalyst (e.g., 4-dimethylaminopyridine) to give compound VI.

Conventional methods and/or techniques of separation and purification known to one of ordinary skill in the art can be used to isolate the compounds of the present invention, as well as the various intermediates related thereto. Such techniques will be well-known to one of ordinary skill in the art and may include, for example, all types of chromatography (high pressure liquid chromatography (HPLC), column chromatography using common adsorbents such as silica gel, and thin-layer chromatography), recrystallization, and differential (i.e., liquid-liquid) extraction techniques.

The compounds of the present invention may be isolated and used per se or in the form of its pharmaceutically acceptable salt, solvate and/or hydrate. The term “salts” refers to inorganic and organic salts of a compound of the present invention. These salts can be prepared in situ during the final isolation and purification of a compound, or by separately reacting the compound or prodrug with a suitable organic or inorganic acid and isolating the salt thus formed. Representative salts include the hydrobromide, hydrochloride, hydroiodide, sulfate, hydrogen sulfate, bisulfate, nitrate, acetate, trifluoroacetate, oxalate, besylate, palmitiate, pamoate, malonate, stearate, laurate, malate, borate, benzoate, lactate, phosphate, hydrogen phosphate, dihydrogen phosphate, hexafluorophosphate, mandelate, methanesulfonate (mesylate), ethanesulfonate, p-toluenesulfonate (tosylate) benzene sulfonate, formate, citrate, maleate, fumarate, succinate, tartrate, naphthylate, mesylate, glucoheptonate, lactobionate, and laurylsulphonate, isonicotinate, salicylate, pantothenate, bitartrate, ascorbate, gentisinate, gluconate, glucaronate, saccharate, benzoate, glutamate, and pamoate (i.e., 1,1′-methylene-bis-(2-hydroxy-3-naphthoate)) salts. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

The term “prodrug” means a compound that is transformed in vivo to yield a compound of Formula (I) or (II). The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

Consequently, the present invention also encompasses pharmaceutical compositions containing prodrugs of compounds of the invention. Compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

For example, if a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound of the present invention contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a compound of the present invention incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY′ wherein Y¹ is H, (C₁-C₆)alkyl or benzyl, —C(OY₀)Y₁ wherein Y₀ is (C₁-C₄) alkyl and Y₁ is (C₁-C₆)alkyl, carboxy(C₁-C₆)alkyl, amino(C₁-C₄)alkyl or mono-N— or di-N,N-(C₁-C₆)alkylaminoalkyl, —C(Y₂)Y₃ wherein Y₂ is H or methyl and Y₃ is mono-N— or di-N,N-(C₁-C₆)alkylamino, morpholino, piperidin-1-yl or pyrrolidin-1-yl.

In certain combination therapies with other lipid-lowering agents, such as those described hereinbelow, e.g., HMG CoA reductase inhibitors, HMG CoA synthetase inhibitors, ACAT inhibitors, squalene synthetase inhibitors, etc., a compound of the present invention may further comprise a prodrug which comprises a compound of the present invention in a hydrolyzable linkage to another agent. Di-ester linkages, for example, are particularly useful for this purpose, i.e., the prodrug is in the form A¹-C(O)O-L′-O(O)C-A², wherein A¹ and A² are the two agents, L¹ is a linker such as a methylene or other (C₁-C₆) alkylene group (alone or further comprising a phenyl or benzyl group). The two agents may both be a compound of the present invention, or one may be another agent useful for treating, e.g., obesity, as described hereinbelow. See, e.g., U.S. Pat. No. 4,342,772-penicillins in di-ester linkages with β-lactamase inhibitors. Accordingly, a compound of the present invention having an available carboxylic acid group provides just one convenient means of producing combination prodrugs of the compound of the invention, which are encompassed by the present invention. Typically, the acidic conditions of the gastrointestinal tract, or enzymes localized in the cells thereof cause the hydrolysis of the prodrug, releasing both agents.

The compounds of the present invention may contain asymmetric or chiral centers, and, therefore, exist in different stereoisomeric forms. It is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans-forms, as well as mixtures, are embraced within the scope of the invention.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers or by resolution of the racemic form by recrystallization techniques, by synthesis from optically-active starting materials, by chiral synthesis, or by chromatographic separation using a chiral stationary phase. Also, some of the compounds of the present invention may be atropisomers (e.g., substituted biaryls) and are considered as part of this invention. Enantiomers can also be separated by use of a chiral HPLC column.

Furthermore, some compounds may exhibit polymorphism. It is to be understood that the present invention encompasses any and all racemic, optically-active, polymorphic and stereoisomeric forms, or mixtures thereof, which form or forms possess properties useful in the treatment of the conditions discussed herein.

The compounds of the present invention may exist in unsolvated as well as solvated forms with pharmaceutically acceptable solvents such as water, ethanol, and the like, and it is intended that the invention embrace both solvated and unsolvated forms.

It is also possible that the compounds of the present invention may exist in different tautomeric forms, and all such forms are embraced within the scope of the invention. For example, all of the tautomeric forms of the imidazole moiety are included in the invention. Also, for example, all keto-enol and imine-enamine forms of the compounds are included in the invention.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The compounds of the instant invention inhibit or decrease Apo B secretion, likely by the inhibition of MTP, although it may be possible that other mechanisms are involved. The compounds are useful in treating any of the disease states or conditions in which Apo B, serum cholesterol, and/or triglyceride levels are elevated. Thus, the compounds of the present invention (including compositions thereof) are useful for the treatment of conditions including atherosclerosis, pancreatitis, obesity, hypercholesterolemia, hypertriglyceridemia, hyperlipidemia and diabetes. Consequently, the compounds of the present invention (including the compositions and processes used therein) may be used in the manufacture of a medicament for the therapeutic applications described herein. Accordingly, the present invention provides pharmaceutical compositions comprising a therapeutically effective amount of a compound of the invention in combination with a pharmaceutically acceptable excipient, diluent, or carrier.

The present invention also relates to a method for inhibiting or decreasing Apo B secretion in an animal in need thereof which comprises the administration of an Apo B secretion inhibiting or decreasing amount of a compound of the present invention. The invention further provides a method of treating a condition selected from atherosclerosis, pancreatitis, obesity (including appetite suppression, weight loss and reduction in food intake), hypercholesterolemia, hypertriglyceridemia, hyperlipidemia, and diabetes which comprises administering to an animal in need of such treatment a therapeutically effective amount of a compound of the present invention. A preferred subgroup of the conditions described hereinabove is atherosclerosis, obesity, hypercholesterolemia, hypertriglyceridemia, hyperlipidemia, and diabetes.

In one aspect of the present invention, a method of treating obesity (including appetite suppression, weight loss and reduction in food intake) in an animal is provided which comprises administering to an animal in need of such treatment a therapeutically effective amount of a compound of the present, wherein the compound is an intestinal-MTP-selective compound. The ED₂₅ of the compound for the inhibition of intestinal fat absorption is preferably at least 5-fold lower than the ED₂₅ of the compound for the lowering of serum triglycerides. In one embodiment, the ED₂₅ for the inhibition of intestinal fat absorption is at least 10-fold lower than the ED₂₅ of the compound for the lowering of serum triglycerides. In another embodiment, the compound exhibits an ED₂₅ for the inhibition of intestinal fat absorption which is at least 50-fold lower than the ED₂₅ of the compound for the lowering of serum triglycerides.

In this invention, the term “selectivity” refers to a greater effect of a compound in a first assay, compared to the effect of the same compound in a second assay. In the above embodiment of the invention, the first assay is for the ability of the compound to inhibit intestinal fat absorption and the second assay is for the ability of the compound to lower serum triglycerides.

In a preferred embodiment, the ability of the compound to inhibit intestinal fat absorption is measured by the ED₂₅ of the compound in an intestinal fat absorption assay, such that a greater effect of the compound results in the observation of a lower absolute (numerical) value for the ED₂₅. In another preferred embodiment, the ability of the compound to lower serum triglycerides is measured by the ED₂₅ of the compound in a serum triglyceride assay. Again, a greater effect of a compound in the serum triglyceride lowering assay results in the observation of a lower absolute (numerical) value for the ED₂₅. An illustrative example of each assay is provided hereinbelow, but it is to be understood that any assay capable of measuring the effectiveness of a compound in inhibiting intestinal fat absorption, or capable of measuring the effectiveness of a compound in lowering serum triglycerides, is encompassed by the present invention.

Another aspect of the present invention concerns the treatment of diabetes, including impaired glucose tolerance, insulin resistance, insulin dependent diabetes mellitus (Type I) and non-insulin dependent diabetes mellitus (NIDDM or Type II). Also included in the treatment of diabetes are the diabetic complications, such as neuropathy, nephropathy, retinopathy or cataracts. Diabetes can be treated by administering to an animal having diabetes (Type I or Type II), insulin resistance, impaired glucose tolerance, or any of the diabetic complications such as neuropathy, nephropathy, retinopathy or cataracts, a therapeutically effective amount of a compound of the present invention. It is also contemplated that diabetes be treated by administering a compound of the present invention along with other agents that can be used to treat diabetes. Preferably, the diabetes is Type II diabetes.

The present invention also provides a method of treating atherosclerosis; pancreatitis secondary to hypertriglyceridemia; hyperglycemia (1) by causing a reduced absorption of dietary fat through MTP inhibition, (2) by lowering triglycerides through MTP inhibition or (3) by decreasing the absorption of free fatty acids through MTP inhibition; in an animal in need of treatment thereof, which comprises administering to the animal a therapeutically effective amount of the compound of the present invention.

As discussed above, the compounds of the present invention are useful for treating diseases, conditions and/or disorders modulated by MTP inhibitors; therefore, another embodiment of the present invention is a pharmaceutical composition comprising a therapeutically effective amount of a compound of the present invention and a pharmaceutically acceptable excipient, diluent or carrier. Alternatively, a compound of the present invention may be administered in combination with at least one additional pharmaceutical agent (referred to herein as a “combination”) which is also preferably administered in the form of a pharmaceutical composition. A compound of the present invention or a combination can be administered in any conventional oral, rectal, transdermal, parenteral, (for example, intravenous, intramuscular, or subcutaneous) intracisternal, intravaginal, intraperitoneal, intravesical, local (for example, powder, ointment or drop), or buccal, or nasal, dosage form. In the combination aspect of the invention, the compound of the present invention and at least one other pharmaceutical agent may be administered either separately or in the pharmaceutical composition comprising both. It is generally preferred that such administration be oral. However, if the subject being treated is unable to swallow, or oral administration is otherwise impaired or undesirable, parenteral or transdermal administration may be appropriate.

When a combination is administered, such administration can be sequential in time or simultaneous with the simultaneous method being generally preferred. For sequential administration, the combination can be administered in any order. It is generally preferred that such administration be oral. It is especially preferred that such administration be oral and simultaneous. When the combination is administered sequentially, the administration of the compound of the present invention and the additional pharmaceutical agent can be by the same or by different methods.

In a combination, the pharmaceutical composition typically comprises (a) a therapeutically effective amount of a compound of the present invention; (b) a therapeutically effective amount of an additional pharmaceutical agent; and (c) a pharmaceutically acceptable excipient, diluent or carrier. Suitable additional pharmaceutical agents include lipid-lowering agents, cholesterol absorption inhibitors, PPAR inhibitors, CETP inhibitors, HMG-CoA reductase inhibitors, HMG-CoA synthase inhibitors, inhibitors of HMG-CoA reductase gene expression, niacin, antioxidants, ACAT inhibitors, squalene synthetase inhibitors, and anti-obesity agents. A preferred additional agent is selected from lovastatin, simvastatin, pravastatin, fluvastatin, atorvastatin (as used herein, the term “atorvastatin” includes the calcium salt of atorvastatin), rosuvastatin, or rivastatin. A more preferred additional agent is atorvastatin.

When an additional anti-obesity agent is used in the combination, the anti-obesity agent(s) is preferably selected from the group consisting of a cannabinoid antagonists (e.g., rimonabant), peptide YY and agonists thereof (e.g., peptide YY₃₋₃₆), MCR-4 agonists, cholecystokinin-A (CCK-A) agonists, monoamine reuptake inhibitors (such as sibutramine), sympathomimetic agents, β₃ adrenergic receptor agonists, dopamine agonists (such as bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), Neuropeptide-Y antagonists, thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin receptor antagonists, histamine 3 receptor antagonists or inverse agonists, neuromedin U receptor agonists and the like. Other anti-obesity agents, including the preferred agents set forth hereinbelow, are well known, or will be readily apparent in light of the instant disclosure, to one of ordinary skill in the art.

Representative anti-obesity agents for use in the combinations, pharmaceutical compositions, and methods of the invention can be prepared using methods known to one of ordinary skill in the art, for example, sibutramine can be prepared as described in U.S. Pat. No. 4,929,629; bromocriptine can be prepared as described in U.S. Pat. Nos. 3,752,814 and 3,752,888; and orlistat can be prepared as described in U.S. Pat. Nos. 5,274,143; 5,420,305; 5,540,917; and 5,643,874. Rimonabant may be prepared as described in U.S. Pat. No. 5,624,941. All of the above recited U.S. patents are incorporated herein by reference.

Especially preferred are anti-obesity agents selected from the group consisting of orlistat, sibutramine, bromocriptine, ephedrine, leptin, and pseudoephedrine. Preferably, compounds of the present invention and combination therapies are administered in conjunction with exercise and a sensible diet.

The additional anti-obesity agent also includes another MTP/apoB inhibitor. Preferred MTP/apoB inhibitors include (i) BMS-197636, also known as 9-[4-[4-(2,3-dihydro-1-oxo-1H-isoindol-2-yl)-1-piperidinyl]butyl]-N-propyl-9H-fluorene-9-carboxamide; (ii) BMS-200150, also known as 2-[1-(3,3-diphenylpropyl)-4-piperidinyl]-2,3-dihydro-1H-isoindol-1-one; and (iii) BMS 201038, also known as 9-[4-(4-[2-(4-trifluoromethylphenyl)-benzoylamino]piperidin-1-yl)butyl]-N-2,2,2-trifluoroethyl)-9H-fluorene-9-carboxamide; and the pharmaceutically acceptable salts of (i), (ii) and (iii). In another embodiment, the anti-obesity agent is selected from the agents disclosed in European Patent Application Nos. 0 584 446 A2 and 0 643 057 A1, the latter of which discloses certain compounds of the formulas

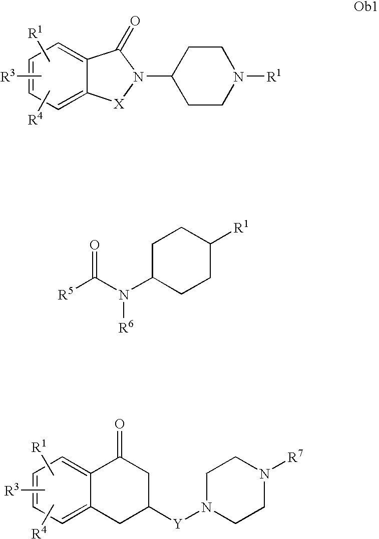

which have utility as inhibitors of MTP, wherein the substituents listed in formula Ob1 are as defined in EP 0 643 057 A1. In another embodiment, the anti-obesity agent is selected from the agents disclosed in European Patent Application No. 1 099 439 A2, which discloses certain compounds of the formula

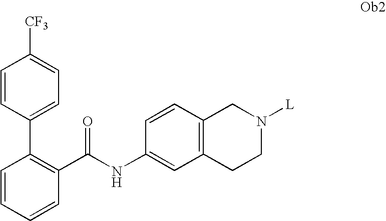

wherein L in formula Ob2 is as defined as in EP 1 099 439 A2.

Preferred compounds of those disclosed in EP1 099 439 A2 are compounds selected from the group consisting of 4′-trifluoromethyl-biphenyl-2-carboxylic acid-(2-butyl-1,2,3,4-tetrahydroisoquinolin-6-yl)-amide and 4′-trifluoromethyl-biphenyl-2-carboxylic acid-(2-(2-acetylaminoethyl)-1,2,3,4-tetrahydroisoquinolin-6-yl)-amide.

The compounds of the present invention may also be administered in combination with a naturally occurring compound that acts to lower plasma cholesterol levels. Such naturally occurring compounds are commonly called nutraceuticals and include, for example, garlic extract, Hoodia plant extracts, and niacin.

Representative agents that can be used to treat diabetes include insulin and insulin analogs (e.g. LysPro insulin); GLP-1 (7-37) (insulinotropin) and GLP-1 (7-36)—NH₂; sulfonylureas and analogs: chlorpropamide, glibenclamide, tolbutamide, tolazamide, acetohexamide, Glypizide®, glimepiride, repaglinide, meglitinide; biguanides: metformin, phenformin, buformin; α2-antagonists and imidazolines: midaglizole, isaglidole, deriglidole, idazoxan, efaroxan, fluparoxan; other insulin secretagogues: linogliride, A-4166; glitazones: ciglitazone, pioglitazone, englitazone, troglitazone, darglitazone, BRL49653; fatty acid oxidation inhibitors: clomoxir, etomoxir; α-glucosidase inhibitors: acarbose, miglitol, emiglitate, voglibose, MDL-25,637, camiglibose, MDL-73,945; β-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine and orlistat; vanadate and vanadium complexes (e.g. Naglivan®) and peroxovanadium complexes; amylin antagonists; glucagon antagonists; gluconeogenesis inhibitors; somatostatin analogs; antilipolytic agents: nicotinic acid, acipimox, WAG 994; and glycogen phosphorylase inhibitors, such as those disclosed in WO 96/39385 and WO 96/39384. Also contemplated in combination with compounds of the invention are pramlintide acetate (Symlin™) and nateglinide. Any combination of agents can be administered as described above.

Specific cholesterol absorption inhibitors and cholesterol biosynthesis inhibitors are described in detail hereinbelow. Additional cholesterol absorption inhibitors are known to those skilled in the art and are described, for example, in PCT WO 94/00480.

Any HMG-CoA reductase inhibitor may be employed as the additional agent in the combination therapy aspect of the instant invention. The term “HMG-CoA reductase inhibitor” refers to a compound which inhibits the biotransformation of hydroxymethylglutaryl-coenzyme A to mevalonic acid as catalyzed by the enzyme HMG-CoA reductase. Such inhibition may be determined readily by one of skill in the art according to standard assays (e.g., Methods of Enzymology, 1981; 71: 455-509 and the references cited therein). A variety of these compounds are described and referenced hereinbelow. U.S. Pat. No. 4,231,938 (the disclosure of which is hereby incorporated by reference) discloses certain compounds isolated after cultivation of a microorganism belonging to the genus Aspergillus, such as lovastatin. Also, U.S. Pat. No. 4,444,784 (the disclosure of which is hereby incorporated by reference) discloses synthetic derivatives of the aforementioned compounds, such as simvastatin. Additionally, U.S. Pat. No. 4,739,073 (the disclosure of which is incorporated herein by reference) discloses certain substituted indoles, such as fluvastatin. Further, U.S. Pat. No. 4,346,227 (the disclosure of which is incorporated herein by reference) discloses ML-236B derivatives, such as pravastatin. In addition, EP 491,226 teaches certain pyridyldihydroxyheptenoic acids, such as rivastatin. Also, U.S. Pat. No. 4,647,576 (the disclosure of which is incorporated herein by reference) discloses certain 6-[2-(substituted-pyrrol-1-yl)alkyl]-pyran-2-ones such as atorvastatin. Other HMG-CoA reductase inhibitors will be known to those skilled in the art.

Any HMG-CoA synthase inhibitor may be used as the second compound in the combination therapy aspect of this invention. The term HMG-CoA synthase inhibitor refers to a compound which inhibits the biosynthesis of hydroxymethylglutaryl-coenzyme A from acetyl-coenzyme A and acetoacetyl-coenzyme A, catalyzed by the enzyme HMG-CoA synthase. Such inhibition may be determined readily by one of skill in the art ac cording to standard assays (e.g., Methods of Enzymology, 35,155-160 (1975) and Methods of Enzymology, 110, 19-26 (1985) and the references cited therein). A variety of these compounds are described and referenced hereinbelow. U.S. Pat. No. 5,120,729 (the disclosure of which is incorporated herein by reference) discloses certain beta-lactam derivatives. U.S. Pat. No. 5,064,856 (the disclosure of which is incorporated herein by reference) discloses certain spiro-lactone derivatives prepared by culturing the microorganism MF5253. U.S. Pat. No. 4,847,271 (the disclosure of which is incorporated herein by reference) discloses certain oxetane compounds such as 11-(3-hydroxymethyl-4-oxo-2-oxetayl)-3,5,7-trimethyl-2,4-undecadienoic acid derivatives. Other HMG-CoA synthase inhibitors will be known to those skilled in the art.

Any compound that decreases HMG-CoA reductase gene expression may be used as the second compound in the combination therapy aspect of this invention. These agents may be HMG-CoA reductase transcription inhibitors that block the transcription of DNA or translation inhibitors that prevent translation of mRNA coding for HMG-CoA reductase into protein.

Such inhibitors may either affect transcription or translation directly, or may be biotransformed into compounds that have the aforementioned attributes by one or more enzymes in the cholesterol biosynthetic cascade or may lead to the accumulation of an isoprene metabolite that has the aforementioned activities. Such regulation is readily determined by those skilled in the art according to standard assays (Methods of Enzymology, 110, 9-19, (1985)). Several such compounds are described and referenced below however other inhibitors of HMG-CoA reductase gene expression will be known to those skilled in the art U.S. Pat. No. 5,041,432 (the disclosure of which is incorporated herein by reference) discloses certain 15-substituted lanosterol derivatives. Other oxygenated sterols that suppress the biosynthesis of HMG-CoA reductase are discussed by E. I. Mercer (Prop. Up. Res., 32, 357416 (1993)).

Any compound having activity as a CETP inhibitor can serve as the additional agent in the combination therapy aspect of the instant invention. The term CETP inhibitor refers to compounds which inhibit the cholesteryl ester transfer protein (CETP) mediated transport of various cholesteryl esters and triglycerides from high density lipoprotein (HDL) to low density lipoprotein (LDL) and very low density lipoprotein (VLDL). A variety of these compounds are described and referenced hereinbelow however other CETP inhibitors will be known to those skilled in the art U.S. Pat. No. 5,512,548 (the disclosure of which is incorporated herein by reference) discloses certain polypeptide derivatives having activity as CETP inhibitors, while certain CETP-inhibitory rosenonolactone derivatives and phosphate-containing analogs of cholesteryl ester are disclosed in J. Antibiot., 49(8): 815-816 (1996), and Bioorg. Med. Chem. Lett; 6, 1951-1954 (1996), respectively.

Any ACAT inhibitor can serve as the additional agent in the combination therapy aspect of this invention. The term ACAT inhibitor refers to compounds which inhibit the intracellular esterification of dietary cholesterol by the enzyme acyl CoA:cholesterol acyltransferase. Such inhibition may be determined readily by one of skill in the art according to standard assays, such as the method of Heider et al. described in Journal of Lipid Research, 24, 1127 (1983). A variety of these compounds are described and referenced hereinbelow however other ACAT inhibitors will be known to those skilled in the art.

U.S. Pat. No. 5,510,379 (the disclosure of which is incorporated herein by reference) discloses certain carboxysulfonates, while WO 96/26948 and WO 96/10559 both disclose urea derivatives having ACAT inhibitory activity.

Any compound having activity as a squalene synthetase inhibitor can serve as the additional agent in the combination therapy aspect of the instant invention. The term squalene synthetase inhibitor refers to compounds that inhibit the condensation of two molecules of farnesylpyrophosphate to form squalene, a reaction that is catalyzed by the enzyme squalene synthetase. Such inhibition is readily determined by those skilled in the art according to standard methodology (Methods of Enzymology, 15, 393-454 (1969) and Methods of Enzymology, 110, 359-373 (1985) and references cited therein). A summary of squalene synthetase inhibitors has been complied (Curr. Op. Ther. Patents, 861-4 (1993).). European Patent Application No. 0 567 026 A1 discloses certain 4,1-benzoxazepine derivatives as squalene synthetase inhibitors and their use in the treatment of hypercholesterolemia and as fungicides. European Patent Application No. 0 645 378 A1 discloses certain seven- or eight-membered heterocycles as squalene synthetase inhibitors and their use in the treatment and prevention of hypercholesterolemia and fungal infections. European Patent Application No. 0 645 377 A1 discloses certain benzoxazepine derivatives as squalene synthetase inhibitors useful for the treatment of hypercholesterolemia or coronary sclerosis. European Patent Application No. 0 611 749 A1 discloses certain substituted amino acid derivatives useful for the treatment of arteriosclerosis. European Patent Application No. 0 705 607 A2 discloses certain condensed seven- or eight-membered heterocyclic compounds useful as antihypertriglyceridemic agents. PCT Publication WO96/09827 discloses certain combinations of cholesterol absorption inhibitors and cholesterol biosynthesis inhibitors including benzoxazepine derivatives and benzothiazepine derivatives. European Patent Application No. 0 071 725 A1 discloses a process for preparing certain optically-active compounds, including benzoxazepine derivatives, having plasma cholesterol and triglyceride lowering activities.

The dosage of the additional pharmaceutical agent will be generally dependent upon a number of factors including the health of the subject being treated, the extent of treatment desired, the nature and kind of concurrent therapy, if any, and the frequency of treatment and the nature of the effect desired. In general, the dosage range of an anti-obesity agent is in the range of from about 0.001 mg to about 500 mg per kilogram body weight of the individual per day, preferably from about 0.01 mg to about 300 mg per kilogram body weight of the individual per day, more preferably from about 0.1 mg to about 100 mg per kilogram body weight of the individual per day. However, some variability in the general dosage range may also be required depending upon the age and weight of the subject being treated, the intended route of administration, the particular anti-obesity agent being administered and the like. The determination of dosage ranges and optimal dosages for a particular patient is also well within the ability of one of ordinary skill in the art having the benefit of the instant disclosure.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above.

Compositions suitable for parenteral injection generally include pharmaceutically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Prevention of microorganism contamination of the compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and gelatin.

Solid dosage forms for oral administration include capsules, tablets, powders, and granules. In such solid dosage forms, a compound of the present invention or a combination is admixed with at least one inert customary pharmaceutical excipient (or carrier) such as sodium citrate or dicalcium phosphate or (a) fillers or extenders (e.g., starches, lactose, sucrose, mannitol, silicic acid and the like); (b) binders (e.g., carboxymethylcellulose, alginates, gelatin, polyvinylpyrrolidone, sucrose, acacia and the like); (c) humectants (e.g., glycerol and the like); (d) disintegrating agents (e.g., agar-agar, calcium carbonate, potato or tapioca starch, alginic acid, certain complex silicates, sodium carbonate and the like); (e) solution retarders (e.g., paraffin and the like); (f) absorption accelerators (e.g., quaternary ammonium compounds and the like); (g) wetting agents (e.g., cetyl alcohol, glycerol monostearate and the like); (h) adsorbents (e.g., kaolin, bentonite and the like); and/or (i) lubricants (e.g., talc, calcium stearate, magnesium stearate, solid polyethylene glycols, sodium lauryl sulfate and the like). In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings and shells, such as enteric coatings and others well known in the art. They may also contain opacifying agents, and can also be of such composition that they release the compound of the present invention and/or the additional pharmaceutical agent in a delayed manner. Examples of embedding compositions that can be used are polymeric substances and waxes. The drug can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the compound of the present invention or the combination, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn germ oil, olive oil, castor oil, sesame seed oil and the like), glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Suspensions, in addition to the compound of the present invention or the combination, may further comprise suspending agents, e.g., ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal or vaginal administration preferably comprise suppositories, which can be prepared by mixing a compound of the present invention or a combination with suitable non-irritating excipients or carriers, such as cocoa butter, polyethylene glycol or a suppository wax which are solid at ordinary room temperature but liquid at body temperature and therefore melt in the rectum or vaginal cavity thereby releasing the active component(s).

Dosage forms for topical administration of the compounds of the present invention and combinations of the compounds of the present invention with an additional pharmaceutical agent(s) may comprise ointments, powders, sprays and inhalants. The drugs are admixed under sterile condition with a pharmaceutically acceptable carrier, and any preservatives, buffers, or propellants that may be required. Ophthalmic formulations, eye ointments, powders, and solutions are also intended to be included within the scope of the present invention.

The compound of the present invention or combination is typically formulated into pharmaceutical dosage forms to provide an easily controllable dosage of the drug and to give the patient an elegant and easily handleable product. The pharmaceutical composition (or formulation) for application may then be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The following paragraphs describe exemplary formulations, dosages, etc. useful for non-human animals. The administration of a compound of the present invention or combination (i.e., a compound of the present invention with at least one additional pharmaceutical agent) can be effected orally or non-orally (e.g., by injection).

An amount of a compound of the present invention (or combination) is administered such that an effective dose is received. Generally, a daily dose that is administered orally to an animal is between about 0.01 and about 1,000 mg/kg of body weight, preferably between about 0.01 and about 300 mg/kg of body weight.

Conveniently, a compound of the present invention (or combination) can be carried in the drinking water so that a therapeutic dosage of the compound is ingested with the daily water supply. The compound can be directly metered into drinking water, preferably in the form of a liquid, water-soluble concentrate (such as an aqueous solution of a water-soluble salt).

Conveniently, a compound of the present invention (or combination) can also be added directly to the feed, as such, or in the form of an animal feed supplement, also referred to as a premix or concentrate. A premix or concentrate of the compound in a carrier is more commonly employed for the inclusion of the agent in the feed. Suitable carriers are liquid or solid, as desired, such as water, various meals such as alfalfa meal, soybean meal, cottonseed oil meal, linseed oil meal, corncob meal and corn meal, molasses, urea, bone meal, and mineral mixes such as are commonly employed in poultry feeds. A particularly effective carrier is the respective animal feed itself; that is, a small portion of such feed. The carrier facilitates uniform distribution of the compound in the finished feed with which the premix is blended. Preferably, the compound is thoroughly blended into the premix and, subsequently, the feed. In this respect, the compound may be dispersed or dissolved in a suitable oily vehicle such as soybean oil, corn oil, cottonseed oil, and the like, or in a volatile organic solvent and then blended with the carrier. It will be appreciated that the proportions of compound in the concentrate are capable of wide variation since the amount of the compound in the finished feed may be adjusted by blending the appropriate proportion of premix with the feed to obtain a desired level of compound.

High potency concentrates may be blended by the feed manufacturer with proteinaceous carrier such as soybean oil meal and other meals, as described above, to produce concentrated supplements, which are suitable for direct feeding to animals. In such instances, the animals are permitted to consume the usual diet. Alternatively, such concentrated supplements may be added directly to the feed to produce a nutritionally balanced, finished feed containing a therapeutically effective level of a compound of the present invention. The mixtures are thoroughly blended by standard procedures, such as in a twin shell blender, to ensure homogeneity.

If the supplement is used as a top dressing for the feed, it likewise helps to ensure uniformity of distribution of the compound across the top of the dressed feed.

Drinking water and feed effective for increasing lean meat deposition and for improving lean meat to fat ratio are generally prepared by mixing a compound of the present invention with a sufficient amount of animal feed to provide from about 10⁻³ to about 500 ppm of the compound in the feed or water.

The preferred medicated swine, cattle, sheep and goat feed generally contain from about 1 to about 400 grams of a compound of the present invention (or combination) per ton of feed, the optimum amount for these animals usually being about 50 to about 300 grams per ton of feed.

The preferred poultry and domestic pet feeds usually contain about 1 to about 400 grams and preferably about 10 to about 400 grams of a compound of the present invention (or combination) per ton of feed.

For parenteral administration in animals, the compounds of the present invention (or combination) may be prepared in the form of a paste or a pellet and administered as an implant, usually under the skin of the head or ear of the animal in which increase in lean meat deposition and improvement in lean meat to fat ratio is sought.

In general, parenteral administration involves injection of a sufficient amount of a compound of the present invention (or combination) to provide the animal with about 0.01 to about 20 mg/kg/day of body weight of the drug. The preferred dosage for poultry, swine, cattle, sheep, goats and domestic pets is in the range of from about 0.05 to about 10 mg/kg/day of body weight of drug.

Paste formulations can be prepared by dispersing the drug in a pharmaceutically acceptable oil such as peanut oil, sesame oil, corn oil or the like.

Pellets containing an effective amount of a compound of the present invention, pharmaceutical composition, or combination can be prepared by admixing a compound of the present invention or combination with a diluent such as carbowax, carnuba wax, and the like, and a lubricant, such as magnesium or calcium stearate, can be added to improve the pelleting process.

It is, of course, recognized that more than one pellet may be administered to an animal to achieve the desired dose level which will provide the increase in lean meat deposition and improvement in lean meat to fat ratio desired. Moreover, implants may also be made periodically during the animal treatment period in order to maintain the proper drug level in the animal's body.

The present invention has several advantageous veterinary features. For the pet owner or veterinarian who wishes to increase leanness and/or trim unwanted fat from pet animals, the instant invention provides the means by which this may be accomplished. For poultry and swine breeders, utilization of the method of the present invention yields leaner animals that command higher sale prices from the meat industry.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 or 500 (available from Varian Inc., Palo Alto, Calif.) at room temperature at 400 and 500 MHz 1H, respectively. Chemical shifts are expressed in parts per million (δ) relative to residual solvent as an internal reference. The peak shapes are denoted as follows: s, singlet; d, doublet; t, triplet; q, quartet; m, multiplet; br s, broad singlet; v br s, very broad singlet; br m, broad multiplet; 2 s, two singlets. In some cases only representative ¹H NMR peaks are given.

Mass spectra were recorded by direct flow analysis using positive and negative atmospheric pressure chemical ionization (APcI) scan modes. A Waters APcI/MS model ZMD mass spectrometer equipped with Gilson 215 liquid handling system was used to carry out the experiments

Mass spectrometry analysis was also obtained by RP-HPLC gradient method for chromatographic separation.

Mobile phase A: 98% water with 2% acetonitrile containing 0.01% formic acid.

B: acetonitrile containing 0.05% formic acid.

Flow rate: 1.0 ml/min

Column: Varian Polaris 2 mm×20 mm 5 μ

Molecular weight identification was recorded by positive and negative electrospray ionization (ESI) scan modes. A Waters/Micromass ESI/MS model ZMD or LCZ mass spectrometer equipped with Gilson 215 liquid handling system and HP 1100 DAD was used to carry out the experiments.

Where the intensity of chlorine or bromine-containing ions are described, the expected intensity ratio was observed (approximately 3:1 for ³⁵CI/³⁷CI-containing ions and 1:1 for ⁷⁹Br/⁸¹Br-containing ions) and only the lower mass ion is given. MS peaks are reported for all examples.

Optical rotations were determined on a PerkinElmer™ 241 polarimeter (available from PerkinElmer Inc., Wellesley, Mass.) using the sodium D line (λ=589 nm) at the indicated temperature and are reported as follows [α]_{D}^{temp}, concentration (c=g/100 ml), and solvent.

Column chromatography was performed with either Baker™ silica gel (40 μm; J. T. Baker, Phillipsburg, N.J.) or Silica Gel 50 (EM Sciences™, Gibbstown, N.J.) in glass columns or in Biotage™ columns (ISC, Inc., Shelton, Conn.) under low nitrogen pressure. Radial chromatography was performed using a Chromatotron™ (Harrison Research).

### Preparation of Key Intermediates

Preparation of Intermediate 4-Amino-3-methyl-benzoic acid methyl ester (I-1b):

A solution of methanol (200 ml) was chilled to 0° C. Acetyl chloride (47.1 g, 600 mmols) was then added dropwise to the stirring mixture to make a 3N HCl solution of methanol. To this solution was added 4-amino-3-methyl-benzoic acid (10.0 g, 66 mmols). The mixture was then refluxed for 5 hours. The mixture was cooled to room temperature and 100 ml of diethyl ether was added. A white precipitate formed and this was collected by suction filtration and washed with ether. The dry product weighed 13.14 g (65 mmols).

Preparation of Intermediate 3-Methyl-4-[(4′-trifluoromethyl-biphenyl-2-carbonyl)-amino]-benzoic Acid Methyl Ester (I-1d):

To a solution of 4-amino-3-methyl-benzoic acid methyl ester I-1b (1.68 g, 10.2 mmols) in methylene chloride (20 ml) was added pyridine (2 ml) and 4′-Trifluoromethyl-biphenyl-2-carbonyl chloride (2.9 g, 10.2 mmols). The solution was stirred at room temperature in an atmosphere of N₂ for 5 hours. The reaction mixture was diluted with methylene chloride (100 ml) and washed with 1N HCl (4×30 ml) and water (20 ml). The organic phase was dried (Na₂SO₄) and concentrated to give a white solid (3.86 g, 9.3 mmols) that was not purified further.

Preparation of Intermediate 3-Methyl-4-[(4′-trifluoromethyl-biphenyl-2-carbonyl)-amino]-benzyl alcohol (I-1e):

To a solution of 3-Methyl-4-[(4′-trifluoromethyl-biphenyl-2-carbonyl)-amino]-benzoic acid methyl ester I-1d (0.51 g, 1.19 mmols) under a nitrogen atmosphere was added LiBH₄ (0.039 g, 1.78 mmols) followed by the dropwise addition of MeOH(0.073 ml, 1.78 mmols). The mixture was then warmed to 65° C. The mixture was stirred at 65° C. for 3 hours. The mixture was poured into 25 ml of cold water. The water was extracted with 2×30 ml of EtOAc. The combined organics were dried (Na₂SO₄) and concentrated to give a viscous oil. Under vacuum the oil became a solid that was used in the next step without purification. Yield=0.461 g, 97%.

Preparation of Intermediate 4′-Trifluoromethyl-biphenyl-2-carboxylic Acid (4-bromomethyl-2-methyl-phenyl)-amide (I-1f):

3-Methyl-4-[(4′-trifluoromethyl-biphenyl-2-carbonyl)-amino]-benzyl alcohol I-1e (0.512 g, 1.33 mmols) was dissolved in 20 ml of anhydrous methylene chloride in an atmosphere of N₂. The solution was cooled to 0° C. and PBr₃ (0.396 g, 1.46 mmols) was added dropwise via syringe. The solution was stirred for 1 hour at 0° C. and for 2 hours at room temperature. The mixture was diluted with 30 ml of methylene chloride and washed with water. The organic phase was dried (Na₂SO₄) and concentrated to give a white solid (0.589 g, 1.31 mmols) that was not purified further.

Preparation of Intermediate 4′-Trifluoromethyl-biphenyl-2-carboxylic Acid (4-azidomethyl-2-methyl-phenyl)-amide (I-1g):

To 4′-Trifluoromethyl-biphenyl-2-carboxylic acid (4-bromomethyl-2-methyl-phenyl)-amide I-1f (16.25 g, 36.2 mmols) in DMF/H₂O (9:1, 100 ml) was added sodium azide (3.5 g, 54.3 mmols). The mixture was stirred at room temperature for 3 hours. Water (200 ml) was added to the reaction mixture to precipitate the product. The white product was collected by filtration, washed with water and dried under vacuum. Yield=14.83 g, 100%.

Preparation of Intermediate 4′-Trifluoromethyl-biphenyl-2-carboxylic Acid (4-aminomethyl-2-methyl-phenyl)-amide (I-1h):

To 4′-Trifluoromethyl-biphenyl-2-carboxylic acid (4-azidomethyl-2-methyl- phenyl)-amide I-1g (1.71 g, 4.16 mmols) dissolved in 1,4-dioxane (15 ml) was added PPh₃ (1.09 g, 4.16 mmols). The reaction mixture was stirred for 12 hours at room temperature and a white precipitate formed. To the mixture was added 1 N NaOH (6 ml) and the mixture was stirred for an additional 3 hours. The mixture was diluted with 25 ml of EtOAc and 25 ml of water. The layers were mixed and the organic layer was saved. The aqueous layer was washed again with EtOAc (25 ml). The combined organics were dried (Na₂SO₄) and concentrated to afford a crude oil. The oil was redissolved in ethyl ether (50 ml) and 5 ml of 4 N HCl in dioxane was added to the solution. The product precipitated out as the HCl salt. The white solid was collected by suction filtration and washed with ethyl ether.

Yield=1.45 g, 3.45 mmols

Preparation of Intermediate (S) ({3-Methyl-4-[(4′-trifluoromethyl-biphenyl-2-carbonyl)-amino]-benzylcarbamoyl}-phenyl-methyl)-carbamic Acid tert-butyl Ester (I-1i):

The HCl salt of 4′-Trifluoromethyl-biphenyl-2-carboxylic acid (4-aminomethyl-2-methyl-phenyl)-amide I-1h (1.45 g, 3.45 mmols), bromo-tris- pyrrolidino-phosphonium hexafluorophosphate (PyBroP) (1.93 g, 4.14 mmols) and Boc protected S-phenyl glycine acid were added to a 50 ml three necked round bottom flask. Anhydrous methylene chloride (15 ml) was added and the mixture was chilled to 0° C. To the chilled solution was added diisopropylethylamine (2.40 ml, 13.8 mmols). The mixture was stirred at 0° C. for 1 hour and at room temperature for 3 hours. The reaction mixture was diluted with chloroform (50 ml) and washed water (1×25 ml). The organic layer was dried (Na₂SO₄) and concentrated to give a viscous oil. The oil was dissolved in a minimal volume of methylene chloride and applied to column of silica gel. The column was eluted with 55% EtOAc in Hexanes. Yield=1.41 g, 2.28 mmols of a white solid.

For those compound of the present invention having a (R) configuration, R- phenyl glycine acid is used instead of S-phenyl glycine acid.

Preparation of Intermediate (S) 4′-Trifluoromethyl-biphenyl-2-carboxylic Acid {4-[(2-amino-2-phenyl-acetylamino)-methyl]-2-methyl-phenyl}-amide (I-1j):

To the Boc compound I-1i (1.41 g, 2.28 mmols) was added an acidic solution of HCl in 1.40 dioxane (4.0 M, 4 ml). The solution was stirred at room temperature for 1 hour. The solvent was evaporated and the solid residue was dried under vacuum. The crusty solid weighed 1.26 g (2.28 mmols).

Preparation of Intermediate (S) 4′-Trifluoromethyl-biphenyl-2-carboxylic Acid {4-[(2-amino-2-phenyl-acetylamino)-methyl]-2-chloro-phenyl}-amide (I-1k):

4′-Trifluoromethyl-biphenyl-2-carboxylic acid {4-[(2-amino-2-phenyl-acetylamino)-methyl]-2-chloro-phenyl}-amide 1-1k was prepared starting with 4-amino-3-chloro-benzoic acid I-1a in a manner analogous to the preparation of 4′-trifluoromethyl-biphenyl-2-carboxylic acid {4-[(2-amino-2-phenyl-acetylamino)-methyl]-2-methyl-phenyl}-amide I-1j above.

Preparation of intermediate III, 4′-Trifluoromethyl-biphenyl-2-carboxylic Acid (5-bromo-3-methyl-pyridin-2-yl)-amide:

To a solution of 2-amino-5-bromo-3-methylpyridine (1-2b) (1.45 g, 7.9 mM) and pyridine (3.2 mL, 39.5 mM) in CH₂Cl₂ (16 mL) was added dropwise a solution of the acid chloride I in CH₂Cl₂ (5 mL). The resulting mixture was stirred at room temperature for 16 h. The mixture was diluted with CH₂Cl₂ and washed with aq. NaHCO₃ (1×25 mL) and water (3×25 mL). The organic fraction was dried (Na₂SO₄), filtered and concentrated. The product was purified by column chromatography (silica gel) eluting with 30% EtOAc in hexanes.

Preparation of Intermediate IV, 4′-Trifluoromethyl-biphenyl-2-carboxylic Acid (5-cyano-3-methyl-pyridin-2-yl)-amide:

Intermediate III (0.69 g, 1.59 mM) was dissolved in NMP (3 mL) in a microwave reaction vial, CuCN (0.358 g, 4.0 mM) was added. The mixture was placed in a microwave and heated to 225° C. for 10 minutes. After cooling the mixture was diluted with EtOAc (20 mL) and a precipitate that had formed was removed by filtration. The filtrate was diluted with EtOAc (50 mL) and washed with water (20 mL) and brine (20 mL). The organic fraction was dried (Na₂SO₄), filtered and concentrated. The product was purified by column chromatography (silica gel) eluting with 50% EtOAc in hexanes.

Preparation of intermediate I-1h.HCl, 4′-Trifluoromethyl-biphenyl-2-carboxylic Acid (5-aminomethyl-3-methyl-pyridin-2-yl)-amide Hydrochloride:

Intermediate IV (0.41 g, 1.07 mM) was dissolved in a 1:1 mixture of MeOH and EtOH (20 mL). To the solution was added concentrated HCl (0.5 mL) and 10% Pd/C (200 mg). The flask was placed on a Parr shaker and shaken under an atmosphere of H₂ (45 psi) for 12 h. The solution was filtered through a pad of celite.

The filtrate was concentrated to provide I-1h.HCl as a colorless solid that was used without further purification.

Preparation of Intermediate VI, (S) 4′-Trifluoromethyl-biphenyl-2-carboxylic Acid {5-[2-amino-2-phenyl-acetylamino)-methyl]-3-methyl-pyridin-2-yl}-amide:

To a solution of I-1h.HCl (0.50 g, 1.30 mM), Boc-Phg-OH (0.325 g, 1.30 mM) and diisopropylethylamine (0.67 mL, 3.9 mM) in CH₂Cl₂ was added DCC (0.268 g, 1.30 mM) and DMAP (0.016 g, 0.13 mM). The mixture was stirred at room temperature for 5 h. The solid that had formed was removed by filtration. The filtrate was concentrated and the residue was purified by column chromatography (silica gel) eluting with 50% EtOAc in hexanes to provide the coupled product (562 mg).

To the product from the above reaction was added a solution of 4M HCl in dioxanes (4 mL). The mixture was stirred at room temperature for 1 h and then concentrated. The residue was taken up in aq. NaHCO₃ (25 mL) and the mixture was extracted with CH₂Cl₂ (3×25 mL). The organic fractions were combined, dried (Na₂SO₄), filtered and concentrated. The product was used as is without further purification.

### Example 1

Preparation of (S) 4′-Trifluoromethyl-biphenyl-2-carboxylic Acid (2-methyl-4-{[2-phenyl-2-(2,2,2-trifluoro-acetylamino)-acetylamino]-methyl}-phenyl)-amide (1A-1):

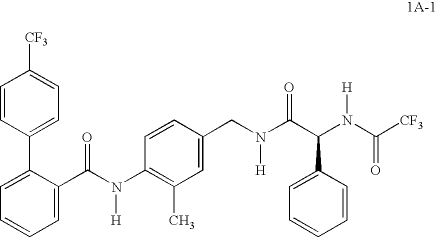

To a chilled (0° C.) solution of the HCl salt of 4′-trifluoromethyl-biphenyl-2-carboxylic acid {4-[(2-amino-2-phenyl-acetylamino)-methyl]-2-methyl-phenyl}-amide I-1h (0.400 g, 0.722 mmols) and PyBroP (0.437 g, 0.939 mmols) was added iPr₂Et (0.372 g, 2.88 mmols) and trifluoroacetic acid (0.082 g, 0.722 mmols). The solution was stirred at 0° C. for 1 hour and at room temperature for 12 hours. A white precipitate settle from the reaction mixture. The precipitate was collected by suction filtration and washed with cold methylene chloride. The precipitate was identified as being pure compound on the basis of spectral and chromatographic data. Yield=0.294 g, 0.479 mmols

The compounds in Table 1 below were prepared using procedures analogous to those described above for the synthesis of Compound IA-1 using the appropriate starting materials which are available commercially, prepared using preparations well-known to those skilled in the art, or prepared in a manner analogous to routes described above for other intermediates. The final products were purified by preparative thin layer chromatography (PTLC) in most cases. In some cases the product did not precipitate from the reaction mixture. In such cases, the products were purified by preparative thin layer chromatography or flash column chromatography on silica gel.

TABLE 1

| Ex. No. | Compound Name | Calc. MW | ESMS (M + 1) |
|---|---|---|---|
| 1A-2 | (S) 4′-Trifluoromethyl-biphenyl-2-carboxylic | 603.647 | 605 |
| | acid (4-{[2-(2-ethoxy-acetylamino)-2- | | |
| | phenyl-acetylamino]-methyl}-2-methyl- | | |
| | phenyl)-amide | | |
| 1A-3 | (S) 4′-Trifluoromethyl-biphenyl-2-carboxylic acid | 589.62 | 590 |
| | (4-{[2-(2-methoxy-acetylamino)-2- | | |
| | phenyl-acetylamino]-methyl}-2-methyl- | | |
| | phenyl)-amide | | |
| 1A-4 | (S) 4′-Trifluoromethyl-biphenyl-2-carboxylic | 603.647 | 604 |
| | acid (4-{[2-(2-hydroxy-2-methyl | | |
| | -propionylamino)-2-phenyl-acetylamino]- | | |
| | methyl}-2-methyl-phenyl)-amide | | |
| 1A-5 | (S) 4′-Trifluoromethyl-biphenyl-2- | 595.574 | 596 |
| | carboxylic acid (4-{[2-(2,2-difluoro- | | |
| | acetylamino)-2-phenyl-acetylamino]-methyl | | |
| | }-2-methyl-phenyl)-amide | | |
| 1A-6 | (S) 4′-Trifluoromethyl-biphenyl-2- | 602.662 | 604 |
| | carboxylic acid (4-{[2-(2-dimethylamino- | | |
| | acetylamino)-2-phenyl-acetylamino]-methyl | | |
| | }-2-methyl-phenyl)-amide | | |
| 1A-7 | (S) 4′-Trifluoromethyl-biphenyl-2-carboxylic | 589.62 | 590 |
| | acid (4-{[2-(3-hydroxy-propionylamino)-2- | | |
| | phenyl-acetylamino]-methyl}-2-methyl- | | |
| | phenyl)-amide | | |
| 1A-8 | (S) 4′-Trifluoromethyl-biphenyl-2- | 675.636 | 694 |
| | carboxylic acid (2-methyl-4-{[2-phenyl- | | (M + 18) |
| | 2-(3,4,5-trifluoro-benzoylamino)- | | |
| | acetylamino]-methyl}-phenyl)-amide | | |
| 1A-9 | (S) 4′-Trifluoromethyl-biphenyl-2- | 678.717 | 696 |
| | carboxylic acid (4-{[2-(4-acetylamino- | | (M + 18) |
| | benzoylamino)-2-phenyl-acetylamino]-methyl | | |
| | }-2-methyl-phenyl)-amide | | |
| 1A-10 | (S) 4′-Trifluoromethyl-biphenyl-2- | 663.703 | 665 |
| | carboxylic acid (4-{[2-(4-acetyl- | | |
| | benzoylamino)-2-phenyl-acetylamino]-methyl | | |
| | }-2-methyl-phenyl)-amide | | |
| 1A-11 | (S) 4′-Trifluoromethyl-biphenyl-2-carboxylic | 644.744 | 646 |
| | acid (4-{[2-(3-diethylamino- | | |
| | propionylamino)-2-phenyl-acetylamino]- | | |
| | methyl}-2-methyl-phenyl)-amide | | |
| 1A-12 | (S) 4′-Trifluoromethyl-biphenyl-2-carboxylic | 634.104 | 635 |
| | acid {2-chloro-4-[(2-hex-3-enoylamino-2- | | |
| | phenyl-acetylamino)-methyl]-phenyl}- | | |
| | amide | | |
| 1A-13 | (S) 4′-Trifluoromethyl-biphenyl-2-carboxylic | 634.104 | 635 |
| | acid (2-chloro-4-{[2-(4-methyl-pent-2- | | |
| | enoylamino)-2-phenyl-acetylamino]- | | |
| | methyl}-phenyl)-amide | | |
| 1A-14 | (S) 4′-Trifluoromethyl-biphenyl-2-carboxylic | 623.08 | 624 |
| | acid (2-chloro-4-{[2-(2-dimethylamino- | | |
| | acetylamino)-2-phenyl-acetylamino]- | | |
| | methyl}-phenyl)-amide | | |
| 1A-15 | (S) 4′-Trifluoromethyl-biphenyl-2-carboxylic | 636.12 | 637 |
| | acid {2-chloro-4-[(2-hexanoylamino-2- | | |
| | phenyl-acetylamino)-methyl]-phenyl}- | | |
| | amide | | |
| 1A-16 | (S) 4′-Trifluoromethyl-biphenyl-2-carboxylic | 632.088 | 633 |
| | acid {2-chloro-4-[(2-hexa-2,4- | | |
| | dienoylamino-2-phenyl-acetylamino)- | | |
| | methyl]-phenyl}-amide | | |
| 1A-17 | (S) 4′-Trifluoromethyl-biphenyl-2-carboxylic | 637.064 | 638 |
| | acid (4-{[2-(2-acetylamino-acetylamino)-2- | | |
| | phenyl-acetylamino]-methyl}-2-chloro- | | |
| | phenyl)-amide | | |
| 1A-18 | (S) 4′-Trifluoromethyl-biphenyl-2-carboxylic | 606.05 | 607 |
| | acid {4-[(2-but-3-enoylamino-2-phenyl- | | |
| | acetylamino)-methyl]-2-chloro-phenyl}- | | |
| | amide | | |
| 1A-19 | (S) 4′-Trifluoromethyl-biphenyl-2-carboxylic | 615.9921 | 617 |
| | acid (2-chloro-4-{[2-(2,2-difluoro- | | |
| | acetylamino)-2-phenyl-acetylamino]- | | |
| | methyl}-phenyl)-amide | | |

The compounds in Table 2 below were prepared in accordance with Scheme II, using procedures analogous to those described above for the synthesis of Intermediates III, IV, I-1h.HCl and VI and Compound 1A-1.

TABLE 2

| | | HPLC | | |
|---|---|---|---|---|
| | | Retention | ESMS | Calc. |
| Ex. No. | Compound Name | time | (m + 1) | MW |
| 1A-20 | (s)4′-Trifluoromethyl-biphenyl-2-carboxylic | 2.74 | 604 | 602.654 |
| | acid {3-methyl-5-[(2-pentanoylamino-2- | | | |
| | phenyl-acetylamino)-methyl]-pyridin-2-yl}- | | | |
| | amide | | | |
| 1A-21 | (s) 4′-Trifluoromethyl-biphenyl-2-carboxylic | 2.83 | 630 | 628.692 |
| | acid (5-{[2-(cyclohexanecarbonyl-amino)-2- | | | |
| | phenyl-acetylamino]-methyl}-3-methyl- | | | |
| | pyridin-2-yl)-amide | | | |
| 1A-22 | (s) 4′-Trifluoromethyl-biphenyl-2-carboxylic | 2.8 | 630 | 628.692 |
| | acid (5-{[2-(2-cyclopentyl-acetylamino)-2- | | | |
| | phenyl-acetylamino]-methyl}-3-methyl- | | | |
| | pyridin-2-yl)-amide | | | |
| 1A-23 | (s) 4′-Trifluoromethyl-biphenyl-2-carboxylic | 2.71 | 604 | 602.654 |
| | acid (3-methyl-5-{[2-(3-methyl- | | | |
| | butyrylamino)-2-phenyl-acetylamino]- | | | |
| | methyl}-pyridin-2-yl)-amide | | | |
| 1A-24 | (s) 4′-Trifluoromethyl-biphenyl-2-carboxylic | 2.62 | 606 | 604.626 |
| | acid (5-{[2-(2-ethoxy-acetylamino)-2- | | | |
| | phenyl-acetylamino]-methyl}-3-methyl- | | | |
| | pyridin-2-yl)-amide | | | |
| 1A-25 | (s) 4′-Trifluoromethyl-biphenyl-2-carboxylic | 2.71 | 604 | 602.654 |
| | acid (3-methyl-5-{[2-(2-methyl- | | | |
| | butyrylamino)-2-phenyl-acetylamino]- | | | |
| | methyl}-pyridin-2-yl)-amide | | | |
| 1A-26 | (s) 4′-Trifluoromethyl-biphenyl-2-carboxylic | 2.51 | 591 | 590.599 |
| | acid (5-{[2-(2-methoxy-acetylamino)-2- | | | |
| | phenyl-acetylamino]-methyl}-3-methyl- | | | |
| | pyridin-2-yl)-amide | | | |
| 1A-27 | (s) 4′-Trifluoromethyl-biphenyl-2-carboxylic | 2.9 | 618 | 616.68 |
| | acid (3-methyl-5-{[2-(3-methyl- | | | |
| | pentanoylamino)-2-phenyl-acetylamino]- | | | |
| | methyl}-pyridin-2-yl)-amide | | | |
| 1A-28 | (s) 4′-Trifluoromethyl-biphenyl-2-carboxylic | 2.9 | 618 | 616.68 |
| | acid (3-methyl-5-{[2-(4-methyl- | | | |
| | pentanoylamino)-2-phenyl-acetylamino]- | | | |
| | methyl}-pyridin-2-yl)-amide | | | |
| 1A-29 | (s) 4′-Trifluoromethyl-biphenyl-2-carboxylic | 1.93 | 605 | 603.642 |
| | acid (5-{[2-(2-dimethylamino-acetylamino)- | | | |
| | 2-phenyl-acetylamino]-methyl}-3-methyl- | | | |
| | pyridin-2-yl)-amide | | | |
| 1A-30 | 5-Oxo-pyrrolidine-2-carboxylic acid [({5- | 2.3 | 630 | 629.636 |
| | methyl-6-[(4′-trifluoromethyl-biphenyl-2- | | | |
| | carbonyl)-amino]-pyridin-3-ylmethyl}- | | | |
| | carbamoyl)-phenyl-methyl]-amide | | | |
| 1A-31 | (s) 4′-Trifluoromethyl-biphenyl-2-carboxylic | 2.35 | 578 | 576.572 |
| | acid (5-{[2-(2-hydroxy-acetylamino)-2- | | | |
| | phenyl-acetylamino]-methyl}-3-methyl- | | | |
| | pyridin-2-yl)-amide | | | |
| 1A-32 | (s) 4′-Trifluoromethyl-biphenyl-2-carboxylic | 2.56 | 620 | 618.653 |
| | acid (5-{[2-(3-ethoxy-propionylamino)-2- | | | |
| | phenyl-acetylamino]-methyl}-3-methyl- | | | |
| | pyridin-2-yl)-amide | | | |
| 1A-33 | (s) 4′-Trifluoromethyl-biphenyl-2-carboxylic | 2.76 | 604 | 602.654 |
| | acid (5-{[2-(2,2-dimethyl-propionylamino)-2- | | | |
| | phenyl-acetylamino]-methyl}-3-methyl- | | | |
| | pyridin-2-yl)-amide | | | |
| 1A-34 | (s) 4′-Trifluoromethyl-biphenyl-2-carboxylic | 2.92 | 631 | 630.707 |
| | acid (5-{[2-(2,2-dimethyl-pentanoylamino)- | | | |
| | 2-phenyl-acetylamino]-methyl}-3-methyl- | | | |
| | pyridin-2-yl)-amide | | | |
| 1A-35 | (s) 4′-Trifluoromethyl-biphenyl-2-carboxylic | 2.64 | 637 | 638.664 |
| | acid (5-{[2-(2-methanesulfonyl- | | (M − 1) | |
| | acetylamino)-2-phenyl-acetylamino]- | | | |
| | methyl}-3-methyl-pyridin-2-yl)-amide | | | |
| 1A-36 | (s) 4′-Trifluoromethyl-biphenyl-2-carboxylic | 2.46 | 641 | 600.638 |
| | acid (5-{[2-(2-cyclopropyl-acetylamino)-2- | | (M + CH3 | |
| | phenyl-acetylamino]-methyl}-3-methyl- | | CN) | |
| | pyridin-2-yl)-amide | | | |
| 1A-37 | 4′-Trifluoromethyl-biphenyl-2-carboxylic | 2.97 | 632 | 630.707 |
| | acid (3-methyl-5-{[2-(2-methyl- | | | |
| | hexanoylamino)-2-phenyl-acetylamino]- | | | |
| | methyl}-pyridin-2-yl)-amide | | | |
| 1A-38 | Tetrahydro-furan-3-carboxylic acid [({5- | 2.46 | 618 | 616.637 |
| | methyl-6-[(4′-trifluoromethyl-biphenyl-2- | | | |
| | carbonyl)-amino]-pyridin-3-ylmethyl}- | | | |
| | carbamoyl)-phenyl-methyl]-amide | | | |
| 1A-39 | Tetrahydro-furan-2-carboxylic acid [({5- | 2.61 | 618 | 616.637 |
| | methyl-6-[(4′-trifluoromethyl-biphenyl-2- | | | |
| | carbonyl)-amino]-pyridin-3-ylmethyl}- | | | |
| | carbamoyl)-phenyl-methyl]-amide | | | |
| 1A-40 | (s) 4′-Trifluoromethyl-biphenyl-2-carboxylic | 2.57 | 635 | 634.655 |
| | acid [5-({2-[2-(2-methoxy-ethoxy)- | | | |
| | acetylamino]-2-phenyl-acetylamino}- | | | |
| | methyl)-3-methyl-pyridin-2-yl]-amide | | | |
| 1A-41 | (s) 4′-Trifluoromethyl-biphenyl-2-carboxylic | 2.31 | 618 | 617.628 |
| | acid (5-{[2-(2-acetylamino-acetylamino)-2- | | | |
| | phenyl-acetylamino]-methyl}-3-methyl- | | | |
| | pyridin-2-yl)-amide | | | |
| 1A-42 | (s) 4′-Trifluoromethyl-biphenyl-2-carboxylic | 2.83 | 618 | 616.684 |
| | acid (5-{[2-(3,3-dimethyl-butyrylamino)-2- | | | |
| | phenyl-acetylamino]-methyl}-3-methyl- | | | |
| | pyridin-2-yl)-amide | | | |
| 1A-43 | (s) 4′-Trifluoromethyl-biphenyl-2-carboxylic | 2.77 | 615 | 614.668 |
| | acid (5-{[2-(cyclopentanecarbonyl-amino)- | | | |
| | 2-phenyl-acetylamino]-methyl}-3-methyl- | | | |
| | pyridin-2-yl)-amide | | | |
| 1A-44 | (s) 4′-Trifluoromethyl-biphenyl-2-carboxylic | 2.75 | 601 | 600.641 |
| | acid (5-{[2-(cyclobutanecarbonyl-amino)-2- | | | |
| | phenyl-acetylamino]-methyl}-3-methyl- | | | |
| | pyridin-2-yl)-amide | | | |
| 1A-45 | (s) 4′-Trifluoromethyl-biphenyl-2-carboxylic | 2.83 | 617 | 616.684 |
| | acid {5-[(2-hexanoylamino-2-phenyl- | | | |
| | acetylamino)-methyl]-3-methyl-pyridin-2-yl}- | | | |
| | amide | | | |
| 1A-46 | (s) 4′-Trifluoromethyl-biphenyl-2-carboxylic | 2.46 | 617 | 616.64 |
| | acid (3-methyl-5-{[2-(4- | | | |
| | oxopentanoylamino)-2-phenyl- | | | |
| | acetylamino]-methyl}-pyridin-2-yl)-amide | | | |
| 1A-47 | (s) 4′-Trifluoromethyl-biphenyl-2-carboxylic | 2.77 | 624 | 622.647 |
| | acid {5-[(2-benzoylamino-2-phenyl- | | | |
| | acetylamino)-methyl]-3-methyl-pyridin-2-yl}- | | | |
| | amide | | | |
| 1A-48 | (s) Thiophene-3-carboxylic acid [({5-methyl- | 2.72 | 629 | 628.673 |
| | 6-[(4′-trifluoromethyl-biphenyl-2-carbonyl)- | | | |
| | amino]-pyridin-3-ylmethyl}-carbamoyl)- | | | |
| | phenyl-methyl]-amide | | | |
| 1A-49 | (s) 5-Methyl-isoxazole-3-carboxylic acid | 2.77 | 628 | 627.623 |
| | [({5-methyl-6-[(4′-trifluoromethyl-biphenyl-2- | | | |
| | carbonyl)-amino]-pyridin-3-ylmethyl}- | | | |
| | carbamoyl)-phenyl-methyl]-amide | | | |
| 1A-50 | (s) 3-Methyl-furan-2-carboxylic acid [({5- | 2.84 | 627 | 626.635 |
| | methyl-6-[(4′-trifluoromethyl-biphenyl-2- | | | |
| | carbonyl)-amino]-pyridin-3-ylmethyl}- | | | |
| | carbamoyl)-phenyl-methyl]-amide | | | |
| 1A-51 | (s) N-[({5-Methyl-6-[(4′-trifluoromethyl- | 2.51 | 634 | 632.639 |
| | biphenyl-2-carbonyl)-amino]-pyridin-3- | | | |
| | ylmethyl}-carbamoyl)-phenyl-methyl]- | | | |
| | succinamic acid methyl ester | | | |
| 1A-52 | 4′-Trifluoromethyl-biphenyl-2-carboxylic | 2.55 | 632 | 630.667 |
| | acid (3-methyl-5-{[2-(2-methyl-4-oxo- | | | |
| | pentanoylamino)-2-phenyl-acetylamino]- | | | |
| | methyl}-pyridin-2-yl)-amide | | | |
| 1A-53 | (s) 4′-Trifluoromethyl-biphenyl-2-carboxylic | 2.51 | 586 | 585.586 |
| | acid (5-{[2-(2-cyano-acetylamino)-2-phenyl- | | | |
| | acetylamino]-methyl}-3-methyl-pyridin-2-yl)- | | | |
| | amide | | | |
| 1A-54 | (s) 4′-Trifluoromethyl-biphenyl-2-carboxylic | 2.77 | 638 | 636.674 |
| | acid {3-methyl-5-[(2-phenyl-2- | | | |
| | phenylacetylamino-acetylamino)-methyl]- | | | |
| | pyridin-2-yl}-amide | | | |
| 1A-55 | (s) N-[({5-Methyl-6-[(4′-trifluoromethyl- | 2.46 | 633 | 632.639 |
| | biphenyl-2-carbonyl)-amino]-pyridin-3- | | | |
| | ylmethyl}-carbamoyl)-phenyl-methyl]- | | | |
| | malonamic acid ethyl ester | | | |
| 1A-56 | (s) 1-Methyl-1H-pyrrole-2-carboxylic acid | 2.73 | 626 | 625.651 |
| | [({5-methyl-6-[(4′-trifluoromethyl-biphenyl-2- | | | |
| | carbonyl)-amino]-pyridin-3-ylmethyl}- | | | |
| | carbamoyl)-phenyl-methyl]-amide | | | |
| 1A-57 | (s) Furan-3-carboxylic acid [({5-methyl-6- | 2.67 | 614 | 612.608 |
| | [(4′-trifluoromethyl-biphenyl-2-carbonyl)- | | | |
| | amino]-pyridin-3-ylmethyl}-carbamoyl)- | | | |
| | phenyl-methyl]-amide | | | |
| 1A-58 | (s) 6-Methyl-N-[({5-methyl-6-[(4′- | 2.41 | 639 | 637.662 |
| | trifluoromethyl-biphenyl-2-carbonyl)-amino]- | | | |
| | pyridin-3-ylmethyl}-carbamoyl)-phenyl- | | | |
| | methyl]-nicotinamide | | | |
| 1A-59 | (s) 2-Methyl-N-[({5-methyl-6-[(4′- | 2.41 | 638 | 637.662 |
| | trifluoromethyl-biphenyl-2-carbonyl)-amino]- | | | |
| | pyridin-3-ylmethyl}-carbamoyl)-phenyl- | | | |
| | methyl]-nicotinamide | | | |
| 1A-60 | (s) 5-Methyl-pyrazine-2-carboxylic acid [({5- | 2.67 | 639 | 638.65 |
| | methyl-6-[(4′-trifluoromethyl-biphenyl-2- | | | |
| | carbonyl)-amino]-pyridin-3-ylmethyl}- | | | |
| | carbamoyl)-phenyl-methyl]-amide | | | |
| 1A-61 | (s) Furan-2-carboxylic acid [({5-methyl-6- | 2.62 | 613 | 612.608 |
| | [(4′-trifluoromethyl-biphenyl-2-carbonyl)- | | | |
| | amino]-pyridin-3-ylmethyl}-carbamoyl)- | | | |
| | phenyl-methyl]-amide | | | |
| 1A-62 | (s) N-[({5-Methyl-6-[(4′-trifluoromethyl- | 2.42 | 624 | 623.635 |
| | biphenyl-2-carbonyl)-amino]-pyridin-3- | | | |
| | ylmethyl}-carbamoyl)-phenyl-methyl]- | | | |
| | isonicotinamide | | | |
| 1A-63 | (s) N-[({5-Methyl-6-[(4′-trifluoromethyl- | 2.47 | 624 | 623.635 |
| | biphenyl-2-carbonyl)-amino]-pyridin-3- | | | |
| | ylmethyl}-carbamoyl)-phenyl-methyl]- | | | |
| | nicotinamide | | | |
| 1A-64 | (s) Pyridine-2-carboxylic acid [({5-methyl-6- | 2.78 | 624 | 623.635 |
| | [(4′-trifluoromethyl-biphenyl-2-carbonyl)- | | | |
| | amino]-pyridin-3-ylmethyl}-carbamoyl)- | | | |
| | phenyl-methyl]-amide | | | |
| 1A-65 | (s) Pyrazine-2-carboxylic acid [({5-methyl- | 2.6 | 625 | 624.623 |
| | 6-[(4′-trifluoromethyl-biphenyl-2-carbonyl)- | | | |
| | amino]-pyridin-3-ylmethyl}-carbamoyl)- | | | |
| | phenyl-methyl]-amide | | | |
| 1A-66 | (s) 6-Methyl-pyridine-2-carboxylic acid [({5- | 2.83 | 639 | 637.662 |
| | methyl-6-[(4′-trifluoromethyl-biphenyl-2- | | | |
| | carbonyl)-amino]-pyridin-3-ylmethyl}- | | | |
| | carbamoyl)-phenyl-methyl]-amide | | | |
| 1A-67 | (s) Thiophene-2-carboxylic acid [({5-methyl- | 2.74 | 629 | 628.673 |
| | 6-[(4′-trifluoromethyl-biphenyl-2-carbonyl)- | | | |
| | amino]-pyridin-3-ylmethyl}-carbamoyl)- | | | |
| | phenyl-methyl]-amide | | | |
| 1A-68 | (s) 4′-Trifluoromethyl-biphenyl-2-carboxylic | 2.83 | 638 | 636.674 |
| | acid (3-methyl-5-{[2-(2-methyl- | | | |
| | benzoylamino)-2-phenyl-acetylamino]- | | | |
| | methyl}-pyridin-2-yl)-amide | | | |
| 1A-69 | (s) 4′-Trifluoromethyl-biphenyl-2-carboxylic | 2.88 | 638 | 636.674 |
| | acid (3-methyl-5-{[2-(3-methyl- | | | |
| | benzoylamino)-2-phenyl-acetylamino]- | | | |
| | methyl}-pyridin-2-yl)-amide | | | |
| 1A-70 | (s) 4′-Trifluoromethyl-biphenyl-2-carboxylic | 2.88 | 638 | 636.674 |
| | acid (3-methyl-5-{[2-(4-methyl- | | | |
| | benzoylamino)-2-phenyl-acetylamino]- | | | |
| | methyl}-pyridin-2-yl)-amide | | | |
| 1A-71 | 4′-Trifluoromethyl-biphenyl-2-carboxylic | 2.27 | 634 | 632.643 |
| | acid (3-methyl-5-{[2-phenyl-2-(2-ureido- | | | |
| | propionylamino)-acetylamino]-methyl}- | | | |
| | pyridin-2-yl)-amide | | | |
| 1A-72 | (s) 4′-Trifluoromethyl-biphenyl-2-carboxylic | 2.61 | 589 | 588.63 |
| | acid {5-[(2-isobutyrylamino-2-phenyl- | | | |
| | acetylamino)-methyl]-3-methyl-pyridin-2-yl}- | | | |
| | amide | | | |
| 1A-73 | (s) 4′-Trifluoromethyl-biphenyl-2-carboxylic | 2.23 | 633 | 632.643 |
| | acid (3-methyl-5-{[2-phenyl-2-(3-ureido- | | | |
| | propionylamino)-acetylamino]-methyl}- | | | |
| | pyridin-2-yl)-amide | | | |
| 1A-74 | (s) 4′-Trifluoromethyl-biphenyl-2-carboxylic | 2.9 | 631 | 630.711 |
| | acid (3-methyl-5-{[2-(5-methyl- | | | |
| | hexanoylamino)-2-phenyl-acetylamino]- | | | |
| | methyl}-pyridin-2-yl)-amide | | | |
| 1A-75 | (s) 4′-Trifluoromethyl-biphenyl-2-carboxylic | 2.69 | 627 | 626.635 |
| | acid (5-{[2-(2-furan-2-yl-acetylamino)-2- | | | |
| | phenyl-acetylamino]-methyl}-3-methyl- | | | |
| | pyridin-2-yl)-amide | | | |
| 1A-76 | (s) Isoxazole-5-carboxylic acid [({5-methyl- | 2.58 | 614 | 613.596 |
| | 6-[(4′-trifluoromethyl-biphenyl-2-carbonyl)- | | | |
| | amino]-pyridin-3-ylmethyl}-carbamoyl)- | | | |
| | phenyl-methyl]-amide | | | |
| 1A-77 | (s) 4′-Trifluoromethyl-biphenyl-2-carboxylic | 2.3 | 632 | 631.655 |
| | acid (5-{[2-(2-acetylamino-propionylamino)- | | | |
| | 2-phenyl-acetylamino]-methyl}-3-methyl- | | | |
| | pyridin-2-yl)-amide | | | |
| 1A-78 | (s) Pyrimidine-5-carboxylic acid [({5-methyl- | 2.46 | 625 | 624.623 |
| | 6-[(4′-trifluoromethyl-biphenyl-2-carbonyl)- | | | |
| | amino]-pyridin-3-ylmethyl}-carbamoyl)- | | | |
| | phenyl-methyl]-amide | | | |
| 1A-79 | 5-Oxo-pyrrolidine-2-carboxylic acid [({5- | 2.38 | 630 | 629.639 |
| | methyl-6-[(4′-trifluoromethyl-biphenyl-2- | | | |
| | carbonyl)-amino]-pyridin-3-ylmethyl}- | | | |
| | carbamoyl)-phenyl-methyl]-mide | | | |

Pharmacological Testing

The utility of the compounds of the present invention in the practice of the instant invention can be evidenced by activity in at least one of the protocols described hereinbelow. Each of the compounds listed in the Examples above were tested in at least one of the following protocols. An EC₅₀ ranging from <1.0 nM to 2.0 nM was observed for the compounds of Examples IA-1 through IA-19. The compounds of Examples IA-20 through IA-79 gave IC₅₀ values ranging from 23 nM to 250 nM in the apo B secretion test below.

### Inhibition of Fat Absorption

Healthy female CF1 mice (Charles River) weighing 18-20 grams upon arrival are employed as test subjects. The mice are housed in groups of 10 in standard caging, and are allowed to acclimate for one week prior to testing. Mice are fasted overnight in a separate procedure room prior to testing. Each treatment group typically consists of 5 mice.

The test compound is preferably provided as a powder in a glass vial. The dosing solution (0.10 ml/25g body weight) administered by oral gavage consists of an emulsion of Miglyol 812 (20%), Cremaphor (5%), Water (75%). An appropriate volume of Miglyol is first added to the test compound, and the vial vortexed for approximately 1 minute. Next, the appropriate volume of Cremaphor is added, and the vial again vortexed as previously. The appropriate volume of water is then added, and the emulsion formed by vortexing and briefly sonicating.

Hamster liquid diet (Bioserve F0739) (dose volume 0.5 ml/25 g body weight) is prepared by adding (for every 10 mL needed) 2.5 grams liquid diet powder, 10 mL water and 5 microcuries glycerol-3H-trioleate (Amersham TRA191) to a laboratory blender. The mixture is then blended at high speed for approximately 1 minute. The liquid diet is stored at 4° C. until use.

Sample tubes are weighed (Falcon 15 ml polypropylene conical). Three milliliters of 2.5N KOH is then added to each tube.

Following overnight fasting, each mouse is dosed (see above volumes) with test compound followed immediately by liquid diet. Positive (a known potent MTP inhibitor) and negative control groups (vehicle) are included in each assay. One scintillation vial is sham dosed every 30 mice in order to determine the activity of the initial bolus.

At two hours post dose the mice are euthanized by carbon dioxide inhalation, the abdominal cavity opened, and the small intestines removed and placed in the KOH conical tube. Each tube is then weighed.

Tubes containing intestines are then placed in a 75° C. water bath for 1.5-2 hours. Following saponification, the tubes are vortexed and 200 μL saponate placed in a 20 mL liquid scintillation vial. Samples are decolorized (for 30 minutes) by adding 200 μL of 30% (w/w) hydrogen peroxide. Each sample is neutralized by the addition of 200 μL of 3N HCL. Ten milliliters of Ready Safe® (Beckman) liquid scintillation fluid are added and the samples were counted on a Beckman Coulter LS 6500 scintillation system.

The calculations are carried out as follows:

weight of saponate=weight of tube (KOH+intestine)−weight of empty tube

saponate fraction=0.22/saponate weight (density of the saponate=1.1 g/mL; therefore the weight of the aliquot is equal to 0.22 g)

total DPM for the entire intestine=DPM of sample/saponate fraction

The initial bolus DPM is calculated by averaging the counts from the sham dosed scintillation vials.

The fraction of bolus recovered from the intestine (percent recovery)=total DPM/bolus count.

Percent recovery from each test group=average of percent recovery from each mouse.

Interpretation of Results:

To compare efficacy of test compounds, an ED₂₅ for intestinal fat absorption is calculated. The (average) percent triglyceride recovery (percent unabsorbed and remaining in the intestine) of the vehicle control group is adjusted to equal 0%, and the (average) percent recovery of the compound control group is adjusted to equal 100%. The same calculations are applied to the percent recovery values obtained for test compounds and an adjusted percent recovery is obtained (% recovery of the test sample—% recovery of vehicle control group/(% recovery of positive control group—% recovery of vehicle control group)). An ED₂₅ is then calculated by plotting a graph of compound concentration vs. adjusted percent recovery.

### Serum Triglyceride Lowering

Healthy female CF1 mice (Charles River) weighing 18-20 grams upon arrival are employed as test subjects. The mice are housed in groups of 10 in standard caging, and were allowed to acclimate for one week prior to testing. Mice are fasted overnight in a separate procedure room prior to testing. Each treatment group typically consists of 10 mice.

The test compound is preferably provided as a powder in a glass vial. The dosing solution (0.250 ml/25 g body weight) administered by oral gavage consists of an emulsion of Miglyol 812 (40%), Cremaphor (10%), Water (50%). An appropriate volume of Miglyol is first added to the test compound, and the vial vortexed for approximately 1 minute. Next, the appropriate volume of Cremaphor is added, and the vial again vortexed as previously. The appropriate volume of water is then added and the emulsion formed by vortexing and briefly sonicating.

Following overnight fasting, each mouse is dosed (see above volumes) with test compound. At 1 hour post dose the mice are euthanized by carbon dioxide inhalation and blood collected for triglyceride quantitation. Serum triglyceride values are quantitated using a calorimetric endpoint assay (Wako Triglyceride E kit # 432-4021) on a Spectra Max 250 plate reader with Softmax Pro software. All samples are run in duplicate.

For comparison of triglyceride values, the percent change from control is calculated. The average triglyceride value of the test compound group is divided by the average triglyceride value of the vehicle group, multiplied by 100 and then subtracted from 100%. The ED₂₅ value is then calculated by plotting a graph of compound concentration versus percent change from control.

The relative values of the ED₂₅ for triglyceride lowering and the ED₂₅ for inhibition of intestinal fat absorption are used as a means to compare selectivity of the test compounds.

APo B Secretion Inhibition/MTP Inhibition Assays

The ability of the compounds of the present invention to inhibit the secretion of apo B and/or inhibit MTP can be determined using the following cell based assay, which measures the secretion of apo B in HepG2 cells.

HepG2 cells (ATCC, HB-8065, Manassas, Va.) are grown in Dulbecco's Modified Eagles Medium plus 10% fetal bovine serum (Growth medium; Gibco, Grand Island, N.Y.) in 96 well culture plates in a humidified atmosphere containing 5% carbon dioxide until they are approximately 70% confluent. Test compounds are dissolved at 10 mM in dimethyl sulfoxide (DMSO). From this stock, the initial dose concentration is prepared in 70% ETOH and subsequent serial dilutions made in 70% ETOH with DMSO at a concentration equivalent to the initial dilution. Dilutions of test compounds are prepared at 100× the desired final concentation and are added in triplicate to separate wells of a 96-well culture plate containing HepG2 cells. Forty hours later, growth medium is collected and assayed by specific enzyme-linked immunosorbent assay (ELISA) for apo B. Inhibitors are identified as compounds that decrease apo B secretion into the medium. The ELISA for apo B is performed as follows. Polyclonal antibody against human apo B (Chemicon, Temecula, Calif.) is diluted 1:1000 in carbonate-bicarbonate buffer (Pierce, Rockford, Ill.) and 100 μL are added to each well of a 96-well plate (NUNC Maxisorb, Rochester, N.Y.). After 5 hour incubation at room temperature, the antibody solution is removed and wells are washed four times with phosphate buffered saline (PBS)/0.05% Tween 20. Non-specific sites on the plastic are blocked by incubating wells for 1-1.5 hours in a solution of 0.5% (w/v) bovine serum albumin (BSA), 0.1% Tween 20 made in PBS. 100 μL of a 1:20 dilution of growth medium from the HepG2 cells (made in 0.004% Tween 20/1% BSA in PBS) are added to each well and incubated for 3 hours at RT. Wells are aspirated and washed four times (0.05% Tween 20 in PBS) prior to adding 100 μL of a 1/1000 dilution (˜5 ug/ml) of the secondary antibody, mouse anti-human apo B (Chemicon, Temecula, Calif.). After 2 hours incubation at room temperature, this solution is aspirated and the wells are again washed 4 times as above. 100 μl of a 1:10,000 dilution (in PBS/1% BSA/0.1% Tween 20) of peroxidase-conjugated affinpure goat anti-mouse IgG (H+L) (Jackson ImunnoResearch Laboratories, Bar Harbor, Me.)) are then added to each well and incubated for 1 hour at room temperature. After aspirating, the wells are washed 4 times as above and 50 μl of 1-step Ultra TMB (tetramethylbenzidine) ELISA reagent (Pierce, Rockford, Ill.) are added to each well and incubated for 5 minutes. The reaction is stopped by the addition of 50 μL of 2M H2SO4 and absorbance of each well is read at 450 nm. Percent inhibition is calculated using absorbance from vehicle treated supernatants minus the absorbance from media alone as the total or 100% value. The percents inhibition at each concentration of test compound are imported into GraphPad Prism software and IC50's determined.

Activity of the compounds of the present invention can also be confirmed when a test compound inhibits MTP activity directly. Inhibition of MTP activity by a compound can be quantitated by observing the inhibition of radiolabeled triglyceride from the donor vesicles to acceptor vesicles in the presence of soluble human MTP. The procedures for preparing MTP are based on the method of Wetterau and Zilversmit (Biochem. Biophys. Acta, 875: 610 (1986)). Briefly, human liver chunks, frozen at −80° C., are thawed on ice, minced, and rinsed several times with ice cold 0.25M sucrose. All subsequent steps are performed on ice. A 50% homogenate in 0.25 M sucrose is prepared using a Potter-Elvehjem Teflon pestle. The homogenate is diluted 1:1 with 0.25 M sucrose and centrifuged at 10,000×g for 20 minutes at 4° C. The pellet is resuspended in sucrose and recentrifuged at 10,000×g for 20 minutes. The supernatants are combined and the microsomes pelleted by centrifugation at 105,000×9 for 75 minutes. The supernatant is decanted and the microsomal pellet is suspended in a minimal volume of 0.25 M sucrose, diluted to 3 ml per gram starting liver weight with 0.15M Tris-HCl, pH 8.0. This suspension is divided into 12 fractions, and centrifuged at 105,000×g for 75 minutes. The supernatants are discarded and the microsomal pellets are stored frozen at −80° C. until needed. For preparation of MTP prior to performing the assay, a thawed pellet is suspended in 12 ml of cold 50 mM Tris-HCl, 50 mM KCl, 5 mM MgCl₂, pH 7.4 and 1.2 ml of a 0.54% deoxycholate (pH 7.4) solution is added slowly with mixing to disrupt the microsomal membrane. After 30 minutes incubation on ice with gentle mixing, the suspension is centrifuged at 105,000×g for 75 minutes. The supernatant containing the soluble MTP protein is dialyzed for 2-3 days with 4 changes of assay buffer (150 mM Tris-HCl, 40 mM NaCl, 1 mM EDTA, 0.02% NaN₃, pH 7.4). The human liver MTP is stored at 4° C. and diluted 1:5 with assay buffer just before use. MTP preparations show no notable loss of transfer activity with storage up to 30 days.

Liposomes are prepared under nitrogen by room temperature, bath sonication of a dispersion of 400 μM egg phosphatidylcholine (PC), 75 μM bovine heart cardiolipin, and 0.82 μM [¹⁴C]-triolein (110 Ci/mol) [New England Nuclear, Boston, Mass.] in assay buffer. The lipids in chloroform are mixed together in the proportions outlined above and then dried under a nitrogen stream before hydrating with assay buffer. Acceptor liposomes are prepared under nitrogen by room temperature bath sonication of a dispersion of 1.2 mM PC, 2.3 μM triolein and 30 pM [³H]-PC (50 Ci/mol) in assay buffer. The donor and acceptor liposomes are centrifuged at 160,000×g for 2 hours at 7° C. The top 80% of the supernatant containing small unilamellar liposomes are carefully removed and stored at 4° C. until used for transfer assays.

MTP activity is measured using a transfer assay that is initiated by mixing donor and acceptor vesicles together with the soluble MTP and test compound. To 100 μl of either a 5% BSA (control) or 5% BSA containing the test compound are added 500 μl assay buffer, 100 μl donor liposomes and 100 μl of diluted MTP protein. After incubation at 37° C. for 45 minutes, triglyceride transfer is terminated by adding 500 μL of a 50% (w/v) diethylaminoethyl (DEAE) cellulose suspension in assay buffer. Following 4 minutes of agitation, the donor liposomes, bound to DEAE cellulose are selectively sedimented by low speed centrifugation (3,000×g; 5 minutes). An aliquot of the supernatant containing the acceptor liposomes is counted and the ³H and ¹⁴C counts are used to calculate the percent recovery of acceptor liposomes and the percent triglyceride transfer using first order kinetics. Inhibition of triglyceride transfer by test compound is manifest as a decrease in ¹⁴C radioactivity compared to controls where no test compound is present.

Reduction of Food Intake Assay

The utility of apo B secretion/MTP inhibitors in the reduction of food intake according to the practice of the methods of the invention is demonstrated according to the following protocol.

Healthy, young adult (1-3 years of age) male and female beagles (Marshall Farms, North Rose, New York, N.Y. 14516) weighing 13-18 kg at the start of the treatment period are employed as test subjects.

The test compound is provided as a powder. The dosing solution, administered by oral gavage, is provided employing a 70/30 polyethylene glycol 400/water solution as the test vehicle. The dosing solution is prepared at 0.1 to 0.5 mg/ml activity so that 1 ml is delivered per kg body weight at dosages of 0.1 to 0.5 mg/kg. Following a seven day acclimation period, a ten day evaluation study is effected.

The study consists of three groups of animals containing 2 male and 2 female dogs, each. Each group of four animals is randomly assigned to receive 0.1, 0.25 or 0.5 mg/kg test compound. On Days 0 to 6, each dog receives the dosing solution administered as a single dose at Time 0 on each dosing day via a feeding tube. This is followed by a 10 ml water rinse to ensure total delivery of dosing solution. Each test animal is permitted ad libitum access to water and IAMS Mini-Chunks® (The Iams Company, P.O. Box 14597, Dayton, Ohio) dry food each day during the study and approximately 0.5-1 hours post-dose.

Reduction in food intake is quantitated by weighing individual food bowls each day prior to feeding and at the end of each 24 hour consumption period during the acclimation period and again during the treatment period. The difference between the weight of the full bowl prior to feeding and the weight of the bowl and amount of food remaining at the end of the 24 hour consumption period represents the reduction in food intake attributable to the test compound.

A reduction in food intake, reduction in body weight, reduction in serum cholesterol and increased fecal fat were observed for the compound of Example 1A-1.

What is claimed is:

1. A compound of Formula (I) 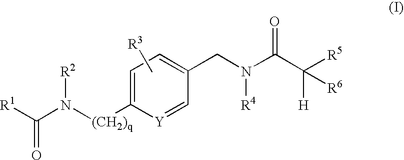
wherein:
R¹ is a group of Formula (IA) having the structure 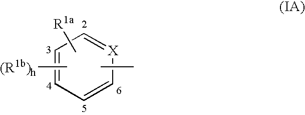
where h is 0 to 3,
X is N or —C(R^{1c})—,
R^{1a} is phenyl, pyridyl, phenyl-Z—, or pyridyl-Z—, where Z is —S(O)ⱼ—, —O—, —(CR^{1a′}R^{1b′})ₖ, or —(O)ₘ(CR^{1a′}R^{1b′})ₖ(O)ₘ(CR^{1a′}R^{1b′})ₖ—, and the phenyl or pyridyl moieties are optionally substituted with 1 to 3 substituents,
R^{1b} and R^{1c} are each independently hydrogen, halo, cyano, nitro, azido, amino, hydroxy, (C₁-C₆)alkyl, (C₂-C₆)alkoxy, methoxy, (C₁-C₆)alkoxy(C₁-C₆)alkyl, mono-, di- or tri-halo(C₂-C₆)alkyl, perfluoro(C₂-C₄)alkyl, trifluoromethyl, trifluoromethyl(C₁-C₅)alkyl, mono-, di- or tri-halo(C₂-C₆)alkoxy, trifluoromethyl(C₁-C₅)alkoxy, (C₁-C₆)alkylthio, hydroxy(C₁-C₆)alkyl, (C₃-C₈)cycloalkyl(CR^{1a′}R^{1b′})ₖ—, (C₂-C₆)alkenyl, (C₂-C₆)alkynyl, (C₁-C₆)alkylamino-, (C₁-C₆)dialkylamino, amino(C₁-C₆)alkyl-, —(CR^{1a′}R^{1b′})ₖNR^{1a′}R^{1b″}, —C(O)NR^{1b′}R^{1b″}-NR^{1b′}C(O)R^{1b″′}, —NR^{1b″}OR^{1b″′}, —CH═NOR^{1b″′}, —NR^{1b′}C(O)OR^{1b″′}, —NR^{1b″}S(O)ⱼR^{1b″′}, —C(O)R^{1b″′}, —C(S)R^{1b″′}, —C(O)OR^{1b″′}, —OC(O)R^{1b″′}, —SO₂NR^{1b′}R^{1b″′}, —S(O)ⱼR^{1b″′}, or —(CR^{1a′}R^{1b′})ₖS(O)ⱼR^{1b″}, where R^{1a′} and R^{1b′} are each independently hydrogen or (C₁-C₆)alkyl, R^{1b″} is H, (C₁-C₆)alkyl, (C₃-C₈)cycloalkyl, —C(O)R^{1b″′}, —C(S)R^{1b″′}, —(CR^{1a′}R^{1b′})ₙO(C₁-C₆ alkyl), —(CR^{1a′}R^{1b′})ₙO(C₁-C₆ alkyl), (CR^{1a′}R^{1b′})ₚC(O)R^{1b″′}, —(CR^{1a′}R^{1b′})ₙR^{1b″′} or —SO₂R^{1b″′}; and
each R^{1b″′} is independently H, (C₁-C₆)alkyl, (C₃-C₈)cycloalkyl, trifluoromethyl, or trifluoromethyl(C₁-C₅)alkyl, wherein the alkyl, moieties of the foregoing R^{1b″′} groups are optionally substituted with 1 to 3 substituents each independently selected from the group consisting of C₁-C₆ alkyl, C₁-C₆ alkoxy, amino, hydroxy, halo, cyano, nitro, trifluoromethyl and trifluoromethoxy, j is 0, 1 or 2, each k is independently an integer from 0 to 6, each m is independently 0 or 1, n is an integer from 1 to 6, and p is an integer from 2 to 5;
R² is H, (C₁-C₆)alkyl, (C₃-C₈)cycloalkyl, —C(O)R^{1b″′}, —C(S)R^{1b″′}, —(CR^{1a′}R^{1b′})ₙO(C₁-C₆ alkyl), —(CR^{1a′}R^{1b′})ₙS(C₁-C₆ alkyl), —(CR^{1a′}R^{1b′})ₚC(O)R^{1b″′}, —(CR^{1a′}R^{1b′})ₚR^{1b″′} or —SO₂R^{1b″′}, or R² taken together with R³ forms a 5- to 6-membered partially saturated heterocyclic ring containing one nitrogen atom within the ring;
q is 0 or 1;
R³ is H, halo, (C₁-C₆)alkyl, or mono-, di- or tri-halo(C₁-C₆)alkyl, or R³ taken together with R² forms a 5- to 6-membered partially saturated heterocyclic ring containing one nitrogen atom within the ring;
Y is N or C(R³);
R⁴ is H, (C₁-C₆)alkyl, (C₃-C₈)cycloalkyl, —C(O)R^{1b″′}, —C(S)R^{1b″′}, —(CR^{1a′}R^{1b′})ₙO(C₁-C₆ alkyl), —(CR^{1a′}R^{1b′})ₙS(C₁-C₆ alkyl), —(CR^{1a′}R^{1b′})ₚC(O)R^{1b″′}, —(CR^{1a′}R^{1b′})ₚR^{1b″′} or —SO₂R^{1b″′};
R⁵ is (C₁-C₆)alkyl, an optionally substituted phenyl, or an optionally substituted heteroaryl;
R⁶ is —NH—C(O)—R a, or —NH—C(O)—OR^{6a}, where
R^{6a} is hydrogen, —(CR^{1a′}R^{1b′})ₙO(C₁-C₆ alkyl), —(CR^{1a′}R^{1b′})ₙS(C₁-C₆ alkyl), —(CR^{1a′}R^{1b′})ₚC(O)R^{1b″″}, —(C₁-C₆)alkylSO₂(C₁-C₆)alkyl, —(C₁-C₆)alkylCO₂(C₁-C₆)alkyl, —CH₂O(C₂-C₆)alkylO(C₁-C₆)alkyl, —(C₁-C₆)alkylN(R^{1a′})CO(C₁-C₆)alkyl, —(C₁-C₆)alkylN(R^{1a′})CON(R^{1a′})(R^{1b′}), —(CR^{1a′}R^{1b′})ₚR^{1b″′}, or —(CH₂)ₛ—R^{6a}, where s is an integer from 0 to 6 and R^{6a} is (C₁-C₆)alkylamino, di(C₁-C₆)alkylamino, or a chemical moiety selected from the group consisting of (C₁-C₆)alkyl, (C₂-C₆)alkenyl, (C₂-C₆)alkynyl, a 3- to 6-membered partially or fully saturated carbocyclic ring, a 3- to 6-membered partially or fully saturated heterocyclic ring, heteroaryl, and phenyl, where said chemical moiety is optionally substituted with 1 to 3 substituents, and wherein any of the above “alkyl”, “alkenyl” or “alkynyl” moieties comprising a CH₃ (methyl), CH₂ (methylene), or CH (methine) group which is not substituted with halogen, SO or SO₂, or attached to a N, O or S atom, optionally bears on said methyl, methylene or methine group a substituent selected from the group consisting of halo, —OR^{1a′}, —SR^{1a′} and —NR^{a′}R^{1b′};
a pharmaceutically acceptable salt thereof, a prodrug of said compound or said salt, or a solvate or hydrate of said compound, said salt or said prodrug.

2. The compound of claim 1 having Formula (II) 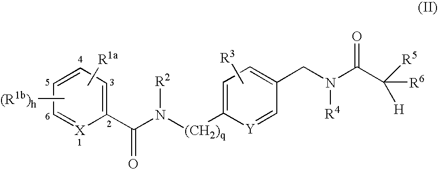
wherein R^{1a}, R^{1b}, h, X, R², q, Y, R³, R⁴, R⁵, and R⁶ are as defined in claim 1;
a pharmaceutically acceptable salt thereof, a prodrug of said compound or said salt, or a solvate or hydrate of said compound, said salt or said prodrug.

3. The compound of claim 2 wherein R^{1a} is attached at the 3 position;
a pharmaceutically acceptable salt thereof, a prodrug of said compound or said salt, or a solvate or hydrate of said compound, said salt or said prodrug.

4. The compound of claim 3 wherein q is 0.

5. The compound of claim 4 wherein X is —C(R^{1c})—;
a pharmaceutically acceptable salt thereof, a prodrug of said compound or said salt, or a solvate or hydrate of said compound, said salt or said prodrug.

6. The compound of claim 5 wherein h is 0 and R^{1c} is hydrogen;
a pharmaceutically acceptable salt thereof, a prodrug of said compound or said salt, or a solvate or hydrate of said compound, said salt or said prodrug.

7. The compound of claim 6 wherein R^{1a} is an optionally substituted phenyl;
a pharmaceutically acceptable salt thereof, a prodrug of said compound or said salt, or a solvate or hydrate of said compound, said salt or said prodrug.

8. The compound of claim 7 wherein R^{1a} is p-trifluoromethylphenyl;
a pharmaceutically acceptable salt thereof, a prodrug of said compound or said salt, or a solvate or hydrate of said compound, said salt or said prodrug.

9. The compound of claim 8 wherein the carbon attached to R⁵ has a (S) configuration.

10. The compound of claim 9 wherein R² and R⁴ are independently H or (C₁-C₆)alkyl.

11. The compound of claim 10 wherein R⁶ is —NH—C(O)—R^{6a};
a pharmaceutically acceptable salt thereof, a prodrug of said compound or said salt, or a solvate or hydrate of said compound, said salt or said prodrug.

12. The compound of claim 11 wherein Y is nitrogen;
a pharmaceutically acceptable salt thereof, a prodrug of said compound or said salt, or a solvate or hydrate of said compound, said salt or said prodrug.

13. The compound of claim 11 wherein Y is C(R³);
a pharmaceutically acceptable salt thereof, a prodrug of said compound or said salt,
or a solvate or hydrate of said compound, said salt or said prodrug.

14. The compound of claim 11 wherein R⁵ is phenyl;
a pharmaceutically acceptable salt thereof, a prodrug of said compound or said salt, or a solvate or hydrate of said compound, said salt or said prodrug.

15. The compound of claim 14 wherein Y is nitrogen;
a pharmaceutically acceptable salt thereof, a prodrug of said compound or said salt, or a solvate or hydrate of said compound, said salt or said prodrug.

16. The compound of claim 14 wherein Y is C(R³);
a pharmaceutically acceptable salt thereof, a prodrug of said compound or said salt, or a solvate or hydrate of said compound, said salt or said prodrug.

17. The compound of claim 16 wherein Y is C(CH₃);
a pharmaceutically acceptable salt thereof, a prodrug of said compound or said salt, or a solvate or hydrate of said compound, said salt or said prodrug.

18. The compound of claim 17 wherein R⁶ is —NHC(O)CF₃;
a pharmaceutically acceptable salt thereof, a prodrug of said compound or said salt, or a solvate or hydrate of said compound, said salt or said prodrug.

19. A compound selected from the group consisting of
(S) 4′-trifluoromethyl-biphenyl-2-carboxylic acid {4-[(2-acetylamino-2-phenyl-acetylamino)-methyl]-2-methyl-phenyl}-amide;
(S) 4′-trifluoromethyl-biphenyl-2-carboxylic acid {2-methyl-4-[(2-phenyl-2-propionylamino-acetylamino)-methyl]-phenyl}-amide;
(S) 4′-trifluoromethyl-biphenyl-2-carboxylic acid {4-[(2-butyrylamino-2-phenyl-acetylamino)-methyl]-2-methyl-phenyl}-amide;
(S) 4′-trifluoromethyl-biphenyl-2-carboxylic acid (2-methyl-4-{[2-phenyl-2-(2,2,2-trifluoro-acetylamino)-acetylamino]-methyl}-phenyl)-amide;
(S) 4′-trifluoromethyl-biphenyl-2-carboxylic acid (2-methyl-4-{[2-phenyl-2-(2-m-tolyl-acetylamino)-acetylamino]-methyl}-phenyl)-amide;
(S) 4′-trifluoromethyl-biphenyl-2-carboxylic acid {4-[(2-phenyl-2-propionylamino-acetylamino)-methyl]-phenyl}-amide;
(S) 4′-trifluoromethyl-biphenyl-2-carboxylic acid {2-methyl-4-[(2-pentanoylamino-2-phenyl-acetylamino)-methyl]-phenyl}-amide;
(S) 4′-trifluoromethyl-biphenyl-2-carboxylic acid {4-[(2-butyrylamino-2-phenyl-acetylamino)-methyl]-2-chloro-phenyl}-amide;
(S) 4′-trifluoromethyl-biphenyl-2-carboxylic acid [4-({2-[2-(3-chloro-phenyl)-acetylamino]-2-phenyl-acetylamino}-methyl)-2-methyl-phenyl]-amide;
(S) 4′-trifluoromethyl-biphenyl-2-carboxylic acid [4-({2-[3-(4-methoxy-phenyl)-propionylamino]-2-phenyl-acetylamino}-methyl)-2-methyl-phenyl]-amide;
(S) 4′-trifluoromethyl-biphenyl-2-carboxylic acid (2-chloro-4{[2-phenyl-2-(2,2,2-trifluoro-acetylamino)-acetylamino]-methyl}-phenyl)-amide;
(S) 4′-trifluoromethyl-biphenyl-2-carboxylic acid {4-[(2-pentanoylamino-2-phenyl-acetylamino)-methyl]-phenyl}-amide;
(S) 4′-trifluoromethyl-biphenyl-2-carboxylic acid [4-({2-[2-(3-fluoro-phenyl)-acetylamino]-2-phenyl-acetylamino}-methyl)-2-methyl-phenyl]-amide;
(S) 4′-trifluoromethyl-biphenyl-2-carboxylic acid [4-({2-[2-(4-ethoxy-phenyl)-acetylamino]-2-phenyl-acetylamino}-methyl)-2-methyl-phenyl]-amide;
(S) 4′-trifluoromethyl-biphenyl-2-carboxylic acid (2-methyl-4-{[2-(2-naphthalen-1-yl-acetylamino)-2-phenyl-acetylamino]-methyl}-phenyl)-amide;
(S) 4′-trifluoromethyl-biphenyl-2-carboxylic acid (4-{[2-(2-methoxy-acetylamino)-2-phenyl-acetylamino]-methyl}-phenyl)-amide;
(S) 4′-trifluoromethyl-biphenyl-2-carboxylic acid (2-methyl-4-{[2-phenyl-2-(4-phenyl-butyrylamino)-acetylamino]-methyl}-phenyl)-amide;
(S) 4′-trifluoromethyl-biphenyl-2-carboxylic acid (4-{[2-(2-methoxy-acetylamino)-2-phenyl-acetylamino]-methyl}-2-methyl-phenyl)-amide;
(S) 4′-trifluoromethyl-biphenyl-2-carboxylic acid (2-chloro-4-{[2-(2-chloro-acetylamino)-2-phenyl-acetylamino]-methyl}-phenyl)-amide; and
(S) 4′-trifluoromethyl-biphenyl-2-carboxylic acid (2-methyl-4-{[2-(2,2,3,3,3-pentafluoro-propionylamino)-2-phenyl-acetylamino]-methyl}-phenyl)-amide;
a pharmaceutically acceptable salt thereof, a prodrug of said compound or said salt, or a solvate or hydrate of said compound, said salt or said prodrug.

20. A compound selected from the group consisting of
(S) 4′-trifluoromethyl-biphenyl-2-carboxylic acid {4-[(2-acetylamino-2-phenyl-acetylamino)-methyl]-2-methyl-phenyl}-amide;
(S) 4′-trifluoromethyl-biphenyl-2-carboxylic acid {4-[(2-butyrylamino-2-phenyl-acetylamino)-methyl]-2-methyl-phenyl}-amide;
(S) 4′-trifluoromethyl-biphenyl-2-carboxylic acid (2-methyl-4-{[2-phenyl-2-(2,2,2-trifluoro-acetylamino)-acetylamino]-methyl}-phenyl)-amide;
(S) 4′-trifluoromethyl-biphenyl-2-carboxylic acid (2-methyl-4-{[2-phenyl-2-(2-m-tolyl-acetylamino)-acetylamino]-methyl}-phenyl)-amide;
(S) 4′-trifluoromethyl-biphenyl-2-carboxylic acid {4-[(2-phenyl-2-propionylamino-acetylamino)-methyl]-phenyl}amide;
(S) 4′-trifluoromethyl-biphenyl-2-carboxylic acid {2-methyl-4-[(2-pentanoylamino-2-phenyl-acetylamino)-methyl]-phenyl}-amide; and
(S) 4′-trifluoromethyl-biphenyl-2-carboxylic acid [4-({2-[2-(3-chloro-phenyl)-acetylamino]-2-phenyl-acetylamino}-methyl)-2-methyl-phenyl]-amide;
a pharmaceutically acceptable salt thereof, a prodrug of said compound or said salt, or a solvate or hydrate of said compound, said salt or said prodrug.

21. A compound having the Formula (1A-1) 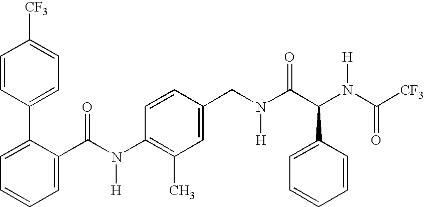
1A-1
a pharmaceutically acceptable salt thereof or a solvate or hydrate of said compound or said salt.

22. A pharmaceutical composition comprising (1) a compound of claim 1, a pharmaceutically acceptable salt thereof, a prodrug of said compound or said salt, or a solvate or hydrate of said compound, said salt or said prodrug; and (2) a pharmaceutically acceptable excipient, diluent, or carrier.

23. The composition of claim 22 further comprising at least one additional pharmaceutical agent selected from a lipid-lowering agent, an anti-obesity agent, a cholesterol absorption inhibitor, a CETP inhibitor, a PPAR inhibitor, an HMG-CoA reductase inhibitor, an HMG-CoA synthase inhibitor, an inhibitor of HMG-CoA reductase gene expression, niacin, an antioxidant, an ACAT inhibitor or a squalene synthetase inhibitor.

24. The pharmaceutical composition of claim 23 wherein said at least one additional agent is selected from lovastatin, simvastatin, pravastatin, fluvastatin, atorvastatin, rosuvastatin, or rivastatin.

25. The pharmaceutical composition of claim 24, wherein said at least one additional agent is atorvastatin.

26. A method of treating obesity in an animal, which comprises administering to an animal in need of such treatment a therapeutically effective amount of a compound of claim 1.

27. A method of treating atherosclerosis; pancreatitis secondary to hypertriglyceridemia or hyperglycemia (1) by causing a reduced absorption of dietary fat through MTP inhibition, (2) by lowering triglycerides through MTP inhibition or (3) by decreasing the absorption of free fatty acids through MTP inhibition in an animal, which comprises administering to an animal in need of such treatment a therapeutically effective amount of a compound of claim 1.

28. A method of treating diabetes in an animal, which comprises administering to an animal in need of such treatment a therapeutically effective amount of a compound of claim 1.

29. A method of treating obesity in an animal, which comprises administering to an animal in need of such treatment a therapeutically effective amount of a compound of claim 1 and one or more anti-obesity agents.

30. The method of claim 29 wherein said anti-obesity agents is selected from the group consisting of cannabinoid antagonists, peptide YY and agonists thereof, MCR-4 agonists, CCK-A agonists, monoamine reuptake inhibitors, sympathomimetic agents, β₃ adrenergic receptor agonists, dopamine agonists, melanocyte-stimulating hormone receptor analogs, 5-HT2c receptor agonists, melanin concentrating hormone antagonists, leptin, leptin analogs, leptin receptor agonists, galanin antagonists, lipase inhibitors, bombesin agonists, neuropeptide-Y antagonists, thyromimetic agents, dehydroepiandrosterone or analogs thereof, glucocorticoid receptor antagonists, orexin receptor antagonists, glucagon-like peptide-1 receptor agonists, ciliary neurotrophic factors, human agouti-related protein antagonists, ghrelin receptor antagonists, histamine 3 receptor antagonists or inverse agonists, and neuromedin U receptor agonists.

31. The use of a compound of claim 1 in the manufacture of a medicament for treating a disease, condition or disorder modulated by inhibition of microsomal triglyceride transfer protein and/or apolipoprotein B secretion in animals.
